(19) United States
(12) Patent Application Publication
(10) Pub. No.: US 2019/0202984 A1
(43) Pub. Date: Jul. 4, 2019

(54) PROCESS OF PREPARING GLATIRAMER ACETATE

(71) Applicant: KinBio Ltd., Rechovot (IL)

(72) Inventor: Joseph Y. KLEIN, Haifa (IL)

(73) Assignee: KinBio Ltd., Rechovot (IL)

(21) Appl. No.: 15/860,738

(22) Filed: Jan. 3, 2018

(51) Int. Cl. C08G 69/04; A61K 31/785; C08G 69/10; C08G 69/48

(52) U.S. Cl. CPC C08G 69/04; C08G 69/48; C08G 69/10; A61K 31/785

(57) ABSTRACT

Disclosed herein is a process of preparing a polypeptide copolymer by polymerizing a mixture comprising N-carboxyanhydrides of alanine, tyrosine, carboxylate-protected glutamate and an amine-protected lysine, to form a protected polypeptide copolymer, which is contacted with a bromine scavenger to form a mixture; subsequently contacting the mixture with a solution of hydrogen bromide in acetic acid, to deprotect carboxylate-protected glutamate; followed by deprotection of the amine-protected lysine. Further disclosed herein is process of deprotecting carboxylate-protected glutamate in a protected polypeptide copolymer described herein by contacting the protected polypeptide copolymer with a bromine scavenger, and subsequently with hydrogen bromide in acetic acid. Further disclosed herein are polypeptide copolymers preparable according to a process as described herein, pharmaceutical compositions comprising same, and methods utilizing same for treating a medical condition.

### FIELD AND BACKGROUND OF THE INVENTION

The present invention, in some embodiments thereof, relates to chemical synthesis and, more particularly, but not exclusively, to a novel process of preparing glatiramer acetate and chemically-related polymeric compounds.

Glatiramer acetate (also referred to in the art as “copolymer-1”) is a random polypeptide copolymer of the amino acids glutamate, lysine, alanine and tyrosine, which is used as an immunomodulator drug for treating multiple sclerosis.

Glatiramer acetate is typically prepared by polymerizing N-carboxyanhydrides of tyrosine, alanine, γ-benzyl glutamate and N-trifluoroacetyl lysine.

U.S. Pat. No. 5,800,808 describes copolymer-1 having a molecular weight of about 5 to 9 kDa (and substantially free of copolymer-1 species having a molecular weight of above 40 kDa) and reports that it is less toxic than copolymer-1 having higher molecular weights. U.S. Pat. No. 5,800,808 further describes the manufacture of copolymer-1 having a molecular weight of about 5 to 9 kDa by polymerizing N-carboxyanhydrides of tyrosine, alanine, γ-benzyl glutamate and N-trifluoroacetyl lysine, to form protected glatiramer acetate; deprotecting the protected glatiramer acetate with a solution of hydrobromic acid in acetic acid, which removes the benzyl protecting group from the glutamate residues and cleaves the polymer to smaller polypeptides, resulting in trifluoroacetyl copolymer-1; and reacting the trifluoroacetyl copolymer-1 with aqueous piperidine to form the copolymer-1.

U.S. Pat. No. 7,495,072 describes the use of a solution of hydrobromic acid in acetic acid which comprises less than 0.5% of free bromine and less than 1000 ppm of metal ion impurities, to form trifluoroacetyl glatiramer acetate. U.S. Pat. No. 7,495,072 further describes treating a solution of hydrobromic acid in acetic acid with a bromine scavenger such as phenol in a non-metallic reactor so as to prepare a treated hydrobromic acid in acetic acid solution with reduced levels of free bromine and metal ion impurities, the use of which is reported therein to result in reduced levels of bromotyrosine residues in the product.

U.S. Pat. No. 7,560,100 describes removing a benzyl protecting group from a polypeptide by contacting the polypeptide with a hydrogen bromide and acetic acid solution at a temperature of 17 to 23° C. for 7 to 18 hours.

Additional background art includes U.S. Pat. Nos. 3,849,550, 5,981,589, 6,048,898 and 7,049,399, and U.S. Patent Application Publication Nos. 2008/0118553 and 2007/0059798.

### SUMMARY OF THE INVENTION

According to an aspect of some embodiments of the invention, there is provided a process of preparing a polypeptide copolymer of alanine, glutamic acid, lysine and tyrosine, or a pharmaceutically acceptable salt thereof, the process comprising:
(a) polymerizing a mixture comprising N-carboxyanhydrides of alanine, tyrosine, carboxylate-protected glutamate and an amine-protected lysine, to form a protected polypeptide copolymer of alanine, tyrosine, carboxylate-protected glutamate, and amine-protected lysine;
(b) contacting the protected polypeptide copolymer with a bromine scavenger to form a mixture of the protected polypeptide copolymer and the bromine scavenger;
(c) subsequent to (b), contacting the mixture with a solution of hydrogen bromide in acetic acid, to deprotect carboxylate-protected glutamate residues in the protected polypeptide copolymer, thereby forming a partially protected polypeptide copolymer of alanine, glutamic acid, tyrosine and amine-protected lysine; and
(d) reacting the partially protected polypeptide copolymer under conditions which effect deprotection of the amine-protected lysine, to form the polypeptide copolymer of alanine, glutamic acid, lysine and tyrosine, or a pharmaceutically acceptable salt thereof.

According to an aspect of some embodiments of the invention, there is provided a polypeptide copolymer of alanine, glutamic acid, lysine and tyrosine, or a pharmaceutically acceptable salt thereof, prepared by a process as described herein in any of the embodiments thereof any combination of these embodiments.

According to an aspect of some embodiments of the invention, there is provided a polypeptide copolymer of alanine, glutamic acid, lysine and tyrosine, or a pharmaceutically acceptable salt thereof, characterized in that a level of brominated tyrosine residues in the polypeptide copolymer is less than 0.03 weight percents of the polypeptide copolymer.

According to an aspect of some embodiments of the invention, there is provided a process of deprotecting carboxylate-protected glutamate residues in a protected polypeptide copolymer of alanine, tyrosine, carboxylate-protected glutamate, and amine-protected lysine, the method comprising:
(i) contacting the protected polypeptide copolymer with a bromine scavenger to form a mixture of the protected polypeptide copolymer and the bromine scavenger; and
(ii) subsequent to (i), contacting the mixture with a solution of hydrogen bromide in acetic acid, thereby deprotecting carboxylate-protected glutamate residues in the protected polypeptide copolymer, thereby forming a partially protected polypeptide copolymer of alanine, glutamic acid, tyrosine and amine-protected lysine.

According to an aspect of some embodiments of the invention, there is provided a pharmaceutical composition comprising the polypeptide copolymer of alanine, glutamic acid, lysine and tyrosine, or a pharmaceutically acceptable salt thereof, prepared according to a process described herein in any of the embodiments thereof and any combination thereof.

According to an aspect of some embodiments of the invention, there is provided a method of treating a medical condition treatable by polypeptide copolymer of alanine, glutamic acid, lysine and tyrosine, or a pharmaceutically acceptable salt thereof, the method comprising administering to a subject in need thereof a therapeutically effective amount of the polypeptide copolymer described herein in any of the embodiments thereof and any combination thereof.

According to an aspect of some embodiments of the invention, there is provided a polypeptide copolymer of alanine, glutamic acid, lysine and tyrosine, or a pharmaceutically acceptable salt thereof, prepared according to a process described herein in any of the embodiments thereof and any combination thereof, for use in a method of treating a medical condition treatable by the polypeptide copolymer, as described herein.

According to some of any of the embodiments of the invention, the bromine scavenger comprises a phenol.

According to some of any of the embodiments of the invention, the phenol comprises unsubstituted phenol.

According to some of any of the embodiments of the invention, the mixture comprises at least 1 gram of the phenol per 15 grams of the protected polypeptide copolymer.

According to some of any of the embodiments of the invention, contacting the mixture with the solution of hydrogen bromide is performed while using a ratio of at least 1 gram of the phenol per 75 grams hydrogen bromide.

According to some of any of the embodiments of the invention, contacting the mixture with the solution of hydrogen bromide is performed while using a ratio of at least 2 grams hydrogen bromide per 1 gram of the mixture.

According to some of any of the embodiments of the invention, a molar ratio of the bromine scavenger to tyrosine residues in the protected polypeptide copolymer is at least 1.5:1.

According to some of any of the embodiments of the invention, contacting the mixture with the solution of hydrogen bromide is performed while using a molar ratio of bromine scavenger to hydrogen bromide which is at least 1:80.

According to some of any of the embodiments of the invention, the solution of hydrogen bromide is not pretreated with a phenol prior to contact with the mixture.

According to some of any of the embodiments of the invention, the carboxylate-protected glutamate is γ-benzyl glutamate.

According to some of any of the embodiments of the invention, the amine-protected lysine is trifluoroacetyl lysine.

According to some of any of the embodiments of the invention, deprotection of the trifluoroacetyl lysine is effected by reaction with aqueous piperidine.

According to some of any of the embodiments of the invention, the mixture of the N-carboxyanhydrides comprises from 40 to 50 weight percents trifluoroacetyl lysine N-carboxyanhydride, from 22.5 to 30 weight percents alanine N-carboxyanhydride, from 15 to 22.5 weight percents γ-benzyl glutamate N-carboxyanhydride, and from 7.5 to 12.5 weight percents tyrosine N-carboxyanhydride.

According to some of any of the embodiments of the invention, the mixture of the N-carboxyanhydrides comprises about 44.6 weight percents trifluoroacetyl lysine N-carboxyanhydride, about 26.9 weight percents alanine N-carboxyanhydride, about 18.8 weight percents γ-benzyl glutamate N-carboxyanhydride, and about 9.7 weight percents tyrosine N-carboxyanhydride.

According to some of any of the embodiments of the invention, the polypeptide copolymer comprises alanine, glutamic acid, lysine and tyrosine residues in molar percentages of from 40 to 50% alanine, from 10 to 18% glutamic acid, from 28 to 36% lysine, and from 7 to 11% tyrosine.

According to some of any of the embodiments of the invention, the polypeptide copolymer comprises alanine, glutamic acid, lysine and tyrosine residues in molar percentages of about 45.1% alanine, about 13.8% glutamic acid, about 32.1% lysine, and about 9.0% tyrosine.

According to some of any of the embodiments of the invention, the polypeptide copolymer or a pharmaceutically acceptable salt thereof is glatiramer acetate.

According to some of any of the embodiments of the invention, the process further comprises purifying the polypeptide copolymer of alanine, glutamic acid, lysine and tyrosine, or a pharmaceutically acceptable salt thereof.

According to some of any of the embodiments of the invention, the purifying comprises ultrafiltration.

According to some of any of the embodiments of the invention, a level of brominated tyrosine residues in the polypeptide copolymer is less than 0.03 weight percents of the polypeptide copolymer.

According to some of any of the embodiments of the invention, a level of brominated tyrosine residues in the polypeptide copolymer is less than 0.0025 weight percents of the polypeptide copolymer.

According to some of any of the embodiments of the invention, contacting the mixture with the solution of hydrogen bromide in acetic acid is effected in a reactor having a volume of at least 100 liters.

According to some of any of the embodiments of the invention, an amount of the polypeptide copolymer of alanine, glutamic acid, lysine and tyrosine is at least 2 kilograms.

According to some of any of the embodiments of the invention, a total amount of the N-carboxyanhydrides in the mixture is at least 5 kilograms.

According to some of any of the embodiments of the invention, the composition further comprises a pharmaceutically acceptable carrier.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING

Some embodiments of the invention are herein described, by way of example only, with reference to the accompanying drawing. With specific reference now to the drawing in detail, it is stressed that the particulars shown are by way of example and for purposes of illustrative discussion of embodiments of the invention. In this regard, the description taken with the drawing makes apparent to those skilled in the art how embodiments of the invention may be practiced.

In the drawings:

FIG. 1 depicts a reactor suitable for large-scale preparation of a polypeptide copolymer according to some embodiments of the invention.

### DESCRIPTION OF SPECIFIC EMBODIMENTS OF THE INVENTION

The present invention, in some embodiments thereof, relates to chemical synthesis and, more particularly, but not exclusively, to a novel process of preparing glatiramer acetate and chemically related polymeric compounds.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The polypeptide copolymer glatiramer acetate is commonly prepared in a protected form, wherein glutamic acid residues are protected by a carboxylate-protecting protecting group such as a benzyl moiety (that is, a form comprising γ-benzyl glutamate residues). The carboxylate-protecting groups (e.g., benzyl moieties) must be cleaved in order to obtain unprotected glutamic acid residues. Hydrobromic acid can cleave carboxylate-protected glutamate (e.g., benzyl glutamate) residues as well as reducing the molecular weight of the copolymer to a desired range, but free bromine (Br₂) present in hydrobromic acid brominates tyrosine residues in glatiramer acetate, resulting in bromotyrosine impurities.

While studying the cleavage of carboxylate-protected glutamate residues by hydrogen bromide, the present inventor has uncovered that bromination of tyrosine residues is reduced considerably when a bromine scavenger such as phenol is added to the protected polypeptide in situ, prior to the addition of hydrogen bromide, that is, by performing the cleavage in a mixture of the protected polypeptide with the bromine scavenger. Such a reaction is simpler and more cost-efficient than pretreating hydrogen bromide with e.g., phenol or any other bromine scavenger (as described, for example in U.S. Pat. No. 7,495,072) so as to obtain hydrogen bromide with reduced amount of free bromine.

Without being bound by any particular theory, it is believed that the bromine scavenger competes with the chemically related tyrosine residues in reacting with whatever free bromine is present in the hydrogen bromide, and thereby inhibits the reaction of tyrosine with bromine.

The process as described herein is surprisingly effective, since it can be effected without removing free bromine from the hydrogen bromide before reacting the hydrogen bromide with the protected polypeptide, while maintaining at least a similar low level of brominated tyrosine residues in the copolymer polypeptide, and even lower levels of brominated tyrosine residues. Furthermore, the process as described herein, by avoiding the need to use hydrogen bromide without free bromine, allows a practitioner to perform a reaction with hydrogen bromide at any time, as the practitioner is not limited by a need to use hydrogen bromide which was pretreated recently and/or by a need to monitor free bromine levels in the hydrogen bromide.

According to an aspect of some embodiments of the present invention, there is provided a novel process of preparing a polypeptide copolymer of alanine, glutamic acid, lysine and tyrosine, or a pharmaceutically acceptable salt thereof.

In some embodiments, the process as described herein results in a polypeptide copolymer of alanine, glutamic acid, lysine and tyrosine, or a pharmaceutically acceptable salt thereof, as defined herein, in which a level of brominated tyrosine residues is less than 0.03, or less than 0.01, or less than 0.001, or less than 0.0005 weight percents of the polypeptide copolymer, as is described in further detail hereinunder.

According to some of any of the embodiments of the present invention, the process comprises:
(a) Polymerizing a mixture comprising N-carboxyanhydrides of alanine, tyrosine, carboxylate-protected glutamate and an amine-protected lysine, to form a protected polypeptide copolymer of alanine, tyrosine, carboxylate-protected glutamate, and amine-protected lysine;
(b) Contacting the protected polypeptide copolymer of alanine, tyrosine, carboxylate-protected glutamate and amine-protected lysine with a bromine scavenger to form a mixture of the protected polypeptide copolymer and the bromine scavenger; and
(c) Subsequent to (b), contacting the mixture with a solution of hydrogen bromide in acetic acid, to deprotect carboxylate-protected glutamate residues in the protected polypeptide copolymer, thereby forming a partially protected polypeptide copolymer of alanine, glutamic acid, tyrosine and amine-protected lysine. According to some of any of the embodiments described herein, the process further comprises, subsequent to (c):
(d) Reacting the partially protected polypeptide copolymer under conditions which effect deprotection of the amine-protected lysine, to form the polypeptide copolymer of alanine, glutamic acid, lysine and tyrosine, or a pharmaceutically acceptable salt thereof.

It is to be appreciated that by contacting the mixture with a solution of hydrogen bromide in acetic acid, the average molecular weight of the polypeptide copolymer is decreased due to polypeptide cleavage induced by the hydrogen bromide. This process is referred to herein as “depolymerization”. The conditions of the polymerization of N-carboxyanhydrides and the conditions (e.g., time period and/or temperature) under which the protected polypeptide copolymer is contacted with the solution of hydrogen bromide are optionally selected such that the degree of depolymerization will result in a desired average molecular weight and/or distribution of molecular weights (e.g., molecular weights such as described by U.S. Pat. No. 5,800,808).

In some of any of the embodiments described herein, the process further comprises purifying the polypeptide copolymer of alanine, glutamic acid, lysine and tyrosine, or a pharmaceutically acceptable salt thereof.

For reasons of simplicity and clarity, the polypeptide copolymer, processes for polymerization, bromine scavenger, hydrogen bromide and its reaction with the polypeptide copolymer, and additional steps in the preparation of the polypeptide copolymer are described separately in different sections hereinafter. It is to be understood that any one of the embodiments described herein regarding one feature may be combined with any one of the embodiments described herein regarding other features, except when the embodiments are incompatible. For example, the polypeptide copolymer according to any one of the embodiments herein regarding the polypeptide copolymer may be combined with the bromine scavenger according to any one of the embodiments described herein regarding the bromine scavenger, and with any one of the embodiments described herein regarding hydrogen bromide (except when incompatible).

### Polypeptide Copolymer:

As used herein, the term “polypeptide” refers to a polymer comprising at least 4 amino acid residues (e.g., amino acid residues described herein), optionally at least 10 amino acid residues, and optionally at least 50 amino acid residues, attached to one another via peptide bonds.

As used herein, the term “polypeptide copolymer” refers to a polypeptide which comprises more than one type of amino acid residue, for example, alanine, lysine, glutamic acid and tyrosine residues, as described herein. The different types of amino acid residues may be configured within the polypeptide in a random or non-random sequence.

Herein, the terms “alanine”, “glutamic acid”, “glutamate”, “lysine” and “tyrosine” may be L-amino acids (L-alanine, L-glutamic acid, L-glutamate, L-lysine and L-tyrosine), D-amino acids (D-alanine, D-glutamic acid, D-glutamate, D-lysine and L-tyrosine) or mixtures of L-amino acids and D-amino acids. In some embodiments, these terms refer to L-amino acids (L-alanine, L-glutamic acid, L-glutamate, L-lysine and L-tyrosine). References below to specific amino acids refer to L-amino acids unless otherwise indicated.

In some embodiments, the polypeptide copolymer comprises residues of amino acids (L-amino acids and/or D-amino acids) other than alanine, glutamic acid, lysine and tyrosine. In some embodiments, at least 50% of the amino acid residues are alanine, glutamic acid, lysine and/or tyrosine residues. In some embodiments, at least 60% of the amino acid residues are alanine, glutamic acid, lysine and/or tyrosine residues. In some embodiments, at least 70% of the amino acid residues are alanine, glutamic acid, lysine and/or tyrosine residues. In some embodiments, at least 80% of the amino acid residues are alanine, glutamic acid, lysine and/or tyrosine residues. In some embodiments, at least 90% of the amino acid residues are alanine, glutamic acid, lysine and/or tyrosine residues. In some embodiments, the amino acid residues consist of alanine, glutamic acid, lysine and/or tyrosine residues. In some embodiments, all of the amino acid residues in the polypeptide copolymer are L-amino acid residues.

It is to be appreciated that the molecules of a copolymer may differ from each other, for example, with respect to sequence, precise percentage of each amino acid type and/or number of amino acid residues therein. Hence, references herein to percentages of amino acid residues in a polypeptide copolymer refer to the average content of polypeptide copolymer molecules.

In some embodiments, a molar percentage of alanine residues in the polypeptide copolymer is from 40 to 50%. In exemplary embodiments, the molar percentage of alanine is about 45.1%.

In some embodiments, a molar percentage of glutamic acid residues in the polypeptide copolymer is from 10 to 18%. In exemplary embodiments, the molar percentage of glutamic acid is about 13.8%.

In some embodiments, a molar percentage of lysine residues in the polypeptide copolymer is from 28 to 36%. In exemplary embodiments, the molar percentage of lysine is about 32.1%.

In some embodiments, a molar percentage of tyrosine residues in the polypeptide copolymer is from 3 to 25%. In some embodiments, a molar percentage of tyrosine residues in the polypeptide copolymer is from 5 to 15%. In some embodiments, a molar percentage of tyrosine residues in the polypeptide copolymer is from 7 to 11%. In exemplary embodiments, the molar percentage of tyrosine is about 9.0%.

In some embodiments, the polypeptide copolymer comprises alanine, glutamic acid, lysine and tyrosine residues in molar percentages of from 40 to 50% alanine, from 10 to 18% glutamic acid, from 28 to 36% lysine, and from 7 to 11% tyrosine. In exemplary embodiments, the polypeptide copolymer comprises (by molar percentages) about 45.1% alanine, about 13.8% glutamic acid, about 32.1% lysine, and about 9.0% tyrosine.

In some embodiments, the polypeptide copolymer is in a form of a pharmaceutically acceptable salt.

As used herein, the phrase “pharmaceutically acceptable salt” refers to a charged species of the parent compound (e.g., a polypeptide copolymer described herein) and at least one counter-ion, which is typically used to modify the solubility characteristics of the parent compound and/or to reduce any significant irritation to an organism by the parent compound, while not abrogating the biological activity and properties of the administered compound.

In the context of the present embodiments, preferably, a pharmaceutically acceptable salt described herein is an acid addition salt which includes lysine residues in which the amine group of lysine is in a form of an ammonium ion, and a counter ion, derived from the selected acid (e.g., acetic acid), that forms a pharmaceutically acceptable salt.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt.

In some embodiments, the acid comprises acetic acid. In some embodiments, the acid consists essentially of acetic acid.

An acetic acid addition salt may be formed by addition of the acetic acid in which the hydrogen bromide is dissolved, as described herein.

In some embodiments, at least a portion of the glutamic acid residues are in the form of glutamate residues.

Herein and in the art, the term “glutamate” refers to the anionic form of glutamic acid, including the anionic form per se, and the anionic form in the context of a salt.

Herein, the term “glutamate” is encompassed by the term “glutamic acid”, and is to be understood as referring to a form of glutamic acid and not to a species distinct from glutamic acid.

In some embodiments, the pharmaceutically acceptable salt of the compounds described herein is a base addition salt (e.g., in addition to being an acid addition salt) which includes glutamate residues in which the carboxylate group of glutamic acid is negatively charged, and a cation counter-ion such as sodium, potassium, ammonium, calcium, magnesium and the like, that forms a pharmaceutically acceptable salt.

In some embodiments, the negative charge of any glutamate residues in the pharmaceutically acceptable salt are offset by a positively charged lysine residue, such that no cation is added to the polypeptide copolymer, and the amount of an acid which is added to form an acid addition salt is reduced due to the presence of glutamate residues.

The amount of counter-ions in the pharmaceutically acceptable salt will depend on the precise amounts of positively lysine residues and negatively charged glutamate residues, as well as the degree to which lysine residues and glutamate residues interact to form an intramolecular salt. For example, positively charged lysine residues will outnumber negatively charged glutamate residues at neutral pH, and therefore a negatively charged counter-ion (e.g., acetate) complements the positive charge of lysine residues which do not have a complementary glutamate residue.

In some embodiments, the pharmaceutically acceptable salt is glatiramer acetate.

As used herein, the term “glatiramer acetate” refers to a salt consisting essentially of a polypeptide copolymer of alanine, glutamic acid, lysine and tyrosine—wherein the alanine, glutamic acid, lysine and tyrosine are present in percentages described herein—and acetate as a counter-ion. The amount of acetate in glatiramer acetate will depend on the precise amounts of lysine residues and glutamic acid residues, as described herein.

In some embodiments, an average molecular weight of the polypeptide copolymer is in a range of from 5 to 9 kDa.

### Polymerization:

The process as described herein for preparing the polypeptide copolymer as described herein starts with polymerizing a mixture of N-carboxyanhydrides of amino acids to be included in the polypeptide copolymer, according to any one of the embodiments described herein. The mixture comprises N-carboxyanhydrides of alanine, tyrosine, carboxylate-protected glutamate and/or an amine protected lysine. For clarity, Scheme 1 below presents the chemical structures of N-carboxyanhydrides of alanine, tyrosine, an exemplary carboxylate-protected glutamate (γ-benzyl glutamate) and an exemplary amine-protected lysine (trifluoroacetyl lysine).

Herein throughout, the terms “polymerization”, “polymerizing” and grammatical diversions thereof are considered interchangeable to “co-polymerizing” and “co-polymerization”, and other grammatical diversions, respectively.

In some embodiments, the γ-benzyl group of the N-carboxyanhydride of γ-benzyl is replaced by another carboxylate-protecting group (e.g., a group recognized in the art as suitable for protecting the carboxylate group of a glutamic acid side chain).

The carboxylate-protected glutamate is preferably an ester of glutamic acid, wherein the carboxylate group of the glutamic side chain is attached via an ester bond to a carboxylate-protecting group. Examples of suitable carboxylate-protecting groups include, without limitation, alkyl groups, optionally unsubstituted alkyl (e.g., t-butyl) and/or alkyl (e.g., methyl) substituted at the 1-position by at least one aromatic group (e.g., substituted or unsubstituted phenyl) and/or heteroatom (e.g., nitrogen) of a heteroalicyclic or heteroaryl group (e.g., N-phthalimido), such as, for example, substituted or unsubstituted benzyl, diphenylmethyl and N-phthalimidomethyl.

The skilled person will be aware of a wide variety of carboxylate-protecting groups known in the art suitable for protecting glutamic acid residues and for being deprotected with hydrogen bromide, as described herein.

It is expected that during the life of a patent maturing from this application many relevant carboxylate-protecting groups will be developed and the scope of the phrases “carboxylate-protected” and “carboxylate-protecting” are intended to include all such new technologies a priori.

In some embodiments, the trifluoroacetyl group of the N-carboxyanhydride of trifluoroacetyl lysine is replaced by another amine-protecting group (e.g., a group recognized in the art as suitable for protecting the amine group of a lysine side chain).

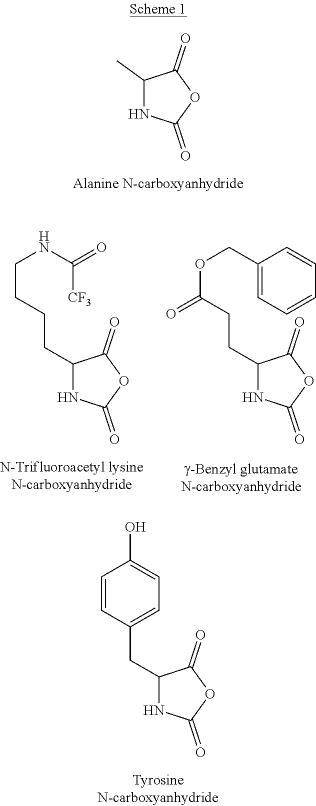

In some embodiments, polymerizing the N-carboxyanhydrides described herein is effected by adding a nucleophile (e.g., an amine) to a solution of the N-carboxyanhydrides in order to initiate polymerization. Diethylamine is an exemplary nucleophile.

In some embodiments, the solvent of the solution of the N-carboxyanhydrides is selected so as to be unreactive towards N-carboxyanhydrides, for example, a solvent which lacks nucleophilic groups such as —OH groups (including water, alcohols and carboxylic acids), —SH groups and amine groups (e.g., ammonia, primary amines or secondary amines). In some embodiments, the solvent is an aprotic solvent. In some embodiments, the aprotic solvent is polar (e.g., water-miscible). In some embodiments, the solvent is an ether. Dioxane is an exemplary solvent.

The skilled person will be capable of recognizing suitable conditions for polymerization of N-carboxyanhydrides to form a polypeptide copolymer. Exemplary conditions are described in the Examples section herein. Suitable conditions are also described, for example, in U.S. Pat. Nos. 7,495,072 and 7,560,100.

Polymerization of N-carboxyanhydrides results in release of carbon dioxide, which may be observed, for example, as bubbling in the reaction mixture. The reaction is optionally allowed to continue at least until release of carbon dioxide ceases.

In some embodiments, the reaction is performed during a time period that ranges from about 1 to 40 hours, optionally from about 3 to 30 hours, optionally from about 6 to 25 hours, and optionally from about 18 to about 24 hours (e.g. optionally about 19 hours, about 20 hours, about 21 hours, about 22 hours, about 23 hours, about 24 hours).

Following polymerization, the obtained protected polypeptide copolymer may optionally be precipitated, for example, by addition of water, optionally water cooled to a temperature of below ambient temperature.

The precipitated protected polypeptide copolymer may be isolated, for example, by centrifugation and/or filtration, and optionally washed. In exemplary embodiments, the partially protected polypeptide copolymer is isolated via centrifugation and is thereafter washed with water.

The skilled person will be capable of determining appropriate amounts of each N-carboxyanhydride in a mixture of N-carboxyanhydrides described herein, so as to obtain, by polymerization, a polypeptide copolymer with a desired composition.

In some embodiments, a weight percentage of alanine N-carboxyanhydride (from the total weight of N-carboxyanhydrides) is in a range of from 22.5 to 30 weight percents. In exemplary embodiments, the percentage of alanine N-carboxyanhydride is about 26.9 weight percents.

In some embodiments, a weight percentage of trifluoroacetyl lysine N-carboxyanhydride (from the total weight of N-carboxyanhydrides) is in a range of from 40 to 50 weight percents. In exemplary embodiments, the percentage of trifluoroacetyl lysine N-carboxyanhydride is about 44.6 weight percents.

In some embodiments, a weight percentage of γ-benzyl glutamate N-carboxyanhydride (from the total weight of N-carboxyanhydrides) is in a range of from 15 to 22.5 weight percents. In exemplary embodiments, the percentage of γ-benzyl glutamate N-carboxyanhydride is about 18.8 weight percents.

In some embodiments, a weight percentage of tyrosine N-carboxyanhydride (from the total weight of N-carboxyanhydrides) is in a range of from 3 to 30 weight percents. In exemplary embodiments, the percentage of tyrosine N-carboxyanhydride is in a range of from 5 to 20 weight percents. In some embodiments, the percentage of tyrosine N-carboxyanhydride is in a range of from 7.5 to 12.5 weight percents. In exemplary embodiments, the percentage of tyrosine N-carboxyanhydride is about 9.7 weight percents.

In some embodiments, the mixture of N-carboxyanhydrides comprises, or consists essentially of, from 40 to 50 weight percents trifluoroacetyl lysine N-carboxyanhydride, from 22.5 to 30 weight percents alanine N-carboxyanhydride, from 15 to 22.5 weight percents γ-benzyl glutamate N-carboxyanhydride, and from 7.5 to 12.5 weight percents tyrosine N-carboxyanhydride. In exemplary embodiments, the mixture of N-carboxyanhydrides comprises about 44.6 weight percents trifluoroacetyl lysine N-carboxyanhydride, about 26.9 weight percents alanine N-carboxyanhydride, about 18.8 weight percents γ-benzyl glutamate N-carboxyanhydride, and about 9.7 weight percents tyrosine N-carboxyanhydride.

### A Mixture of a Polypeptide Copolymer and Bromine Scavenger:

The bromine scavenger according to any one of the embodiments described in this section may be used in combination with a protected polypeptide copolymer according to any one of the embodiments in the section regarding the polypeptide copolymer, and with hydrogen bromide in accordance with any one of the embodiments described in the section regarding hydrogen bromide (except when incompatible).

As used herein, the term “bromine scavenger” refers to a compound which readily reacts with free bromine (Br₂) to produce a less reactive (compared to Br₂) bromine-containing product, for example, by reducing Br₂ to two bromide (Br⁻) ions (e.g., in the form of hydrogen bromide or a bromide salt) and/or by reacting with Br₂ to produce a bromide ion (e.g., in the form of hydrogen bromide or a bromide salt) and a covalently bound bromine atom (e.g., bromine covalently bound to a carbon atom, optionally to a carbon atom in an aromatic ring).

In some embodiments, the bromine scavenger comprises a phenol. In some embodiments, at least 10 weight percents of the bromine scavenger is a phenol. In some embodiments, at least 20 weight percents of the bromine scavenger is a phenol. In some embodiments, at least 30 weight percents of the bromine scavenger is a phenol. In some embodiments, at least 40 weight percents of the bromine scavenger is a phenol. In some embodiments, at least 50 weight percents of the bromine scavenger is a phenol. In some embodiments, at least 60 weight percents of the bromine scavenger is a phenol. In some embodiments, at least 70 weight percents of the bromine scavenger is a phenol. In some embodiments, at least 80 weight percents of the bromine scavenger is a phenol. In some embodiments, at least 90 weight percents of the bromine scavenger is a phenol. In some embodiments, the bromine scavenger consists essentially of a phenol.

As used herein, the term “phenol” encompasses the compound having the formula C₆H₅OH and known in the art as “phenol”, as well as derivatives thereof comprising an —OH group attached to a phenyl ring substituted with one or more substituents, and any combinations thereof. It is to be understood that the singular form “phenol” and phrases such as “a phenol” and “the phenol” encompass one or more phenols (as defined herein), including any combination thereof.

Herein, unsubstituted phenol describes the compound having the formula C₆H₅OH. The term “phenol” as used herein encompasses a phenyl substituted by at least one hydroxyl group, and optionally substituted by one or more additional substituents. Additional substituents can be, for example, alkyl (e.g., methyl, ethyl, propyl), alkenyl (e.g., vinyl), alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, hydrazine, and amino, as these terms are defined herein.

Herein, the term “hydroxy” refers to an —OH group, and the terms “hydroxy” and “—OH” are used interchangeably.

Herein, the terms “phenyl” and “phenyl ring” refer to a six-membered all-carbon ring having a completely conjugated pi-electron system. The aryl group may be substituted or unsubstituted. When substituted, the substituents can be, for example, alkyl (e.g., methyl, ethyl, propyl), alkenyl (e.g., vinyl), alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, hydrazine, and amino, as these terms are defined herein.

In some embodiments of any of the embodiments described herein, the phenol comprises the compound phenol (C₆H₅OH), namely, unsubstituted phenol, wherein the phenyl ring has a single substituent (hydroxy).

In some embodiments of any of the embodiments described herein, at least 10 weight percents of the phenol is unsubstituted phenol (C₆H₅OH). In some embodiments, at least 20 weight percents of the phenol is unsubstituted phenol (C₆H₅OH). In some embodiments, at least 30 weight percents of the phenol is unsubstituted phenol (C₆H₅OH). In some embodiments, at least 40 weight percents of the phenol is unsubstituted phenol (C₆H₅OH). In some embodiments, at least 50 weight percents of the phenol is unsubstituted phenol (C₆H5OH). In some embodiments, at least 60 weight percents of the phenol is unsubstituted phenol (C₆H₅OH). In some embodiments, at least 70 weight percents of the phenol is unsubstituted phenol (C₆H₅OH). In some embodiments, at least 80 weight percents of the phenol is unsubstituted phenol (C₆H₅OH). In some embodiments, at least 90 weight percents of the phenol is unsubstituted phenol (C₆H₅OH).

In some embodiments of any of the embodiments described herein, the phenol consists essentially of unsubstituted phenol (C₆H₅OH).

In some embodiments of any of the embodiments described herein, the phenol comprises a substituted phenol, that is, a compound wherein the phenyl ring has at least one additional substituent in addition to a hydroxy group.

In some embodiments of any of the embodiments described herein, the phenol is substituted by one or more substituents which promote reaction with bromine via electrophilic aromatic substitution. Such substituents are known in the art as “activating groups”. Examples of such substituents include, without limitation, alkyl, alkenyl, alkynyl, cycloalkyl, aryl, heteroaryl, heteroalicyclic, hydroxy, alkoxy, aryloxy, thiohydroxy, thioalkoxy, thioaryloxy, N-amido and amino, as these terms are defined herein.

In some embodiments of any of the embodiments described herein, the phenol is not substituted by halo, sulfinyl, sulfonyl, sulfonate, nitro, phosphonyl, phosphinyl, carbonyl, thiocarbonyl, C-amido, C-carboxy and sulfonamido. Without being bound by any particular theory, it is believed that these substituents are “deactivating groups” which inhibit reaction with bromine via electrophilic aromatic substitution.

In some embodiments of any of the embodiments described herein, the phenol is substituted with one or more hydroxy groups, that is, the phenyl ring is substituted by more than one hydroxy group.

Examples of substituted phenols which may be used according to some embodiments of the invention include, without limitation, o-cresol (2-methylphenol), m-cresol (3-methylphenol), p-cresol (4-methylphenol), 2,6-xylenol (2,6-dimethylphenol), 2,5-xylenol (2,5-dimethylphenol), 2,4-xylenol (2,4-dimethylphenol), 2,3 -xylenol (2,3 -dimethylphenol), 3 ,4-xylenol (3 ,4-dimethylphenol), 3,5-xylenol (3,5-dimethylphenol), catechol (2-hydroxyphenyl), resorcinol (3-hydroxylphenol), hydroquinone (4-hydroxyphenol), guaiacol (2-methoxyphenol), mequinol (4-methoxyphenol), 2-aminophenol, 3-aminophenol, 4-aminophenol, hydroxyquinol (2,4-dihydroxyphenol), phloroglucinol (3,5-dihydroxyphenol) and pyrogallol (2,3-dihydroxyphenol).

In some embodiments of any of the embodiments described herein, the phenyl ring of the phenol is non-substituted at least one position which is an ortho and/or para position with respect to the hydroxy group. In some embodiments, the phenyl ring of the phenol is non-substituted at least two positions which are ortho and/or para with respect to the hydroxy group. In some embodiments, the phenyl ring of the phenol is non-substituted at three positions which are ortho and/or para with respect to the hydroxy group (e.g., one para position and two ortho positions).

Without being bound by any particular theory, it is believed that the ortho and para positions (with respect to the hydroxy group) in a phenol are particularly reactive towards bromine, and absence of a substituent at such a position therefore facilitates reaction between the phenol and bromine.

In some embodiments of any of the embodiments described herein, a bromine scavenger other than a phenol may optionally be used instead of, or in combination with a phenol (e.g., a phenol described herein). In some embodiments, the non-phenol bromine scavenger is a reducing agent, preferably a relatively non-reactive (e.g., mild) reducing agent, so as to minimize any reactions between the bromine scavenger and the protected polypeptide copolymer. Examples of such reducing agents which may be used as bromine scavengers include, without limitation, a sulfite and/or bisulfite salt (e.g., a sodium salt, a calcium salt, a potassium salt), a metabisulfite salt (e.g., potassium metabisulfite, sodium metabisulfite), a thiosulfate salt (e.g., sodium thiosulfate), a thiol, ascorbic acid or a salt thereof (e.g., calcium ascorbate, potassium ascorbate, sodium ascorbate), an ascorbic acid derivative (e.g., ascorbyl palmitate, ascorbyl stearate) or a salt thereof, erythorbic acid or a salt thereof (e.g., sodium erythorbate), a tin(II) salt (e.g., stannous chloride) and aluminum (e.g., aluminum particles).

In some embodiments of any of the embodiments described herein, the bromine scavenger (e.g., phenol) that is contacted with the polypeptide copolymer is devoid of hydrogen bromide and/or free bromine.

By “devoid of” it is meant less than 0.1 weight percents.

In some embodiments, the weight percentage of hydrogen bromide and/or free bromine in the bromine scavenger (e.g., phenol) is no more than 0.03%. In some embodiments, the weight percentage of hydrogen bromide and/or free bromine in the bromine scavenger (e.g., phenol) is no more than 0.01%. In some embodiments, the weight percentage of hydrogen bromide and/or free bromine in the bromine scavenger (e.g., phenol) is no more than 0.003%. In some embodiments, the weight percentage of hydrogen bromide and/or free bromine in the bromine scavenger (e.g., phenol) is no more than 0.001%.

For brevity, the mixture formed by contacting the bromine scavenger (e.g., phenol) and protected polypeptide copolymer as described herein, is referred to herein as “the mixture”.

In some embodiments of any of the embodiments described herein, the mixture of bromine scavenger and polypeptide copolymer comprises at least 1 gram of phenol per 15 grams of polypeptide copolymer. In some embodiments, the mixture comprises at least 1 gram of phenol per 10 grams of polypeptide copolymer. In some embodiments, the mixture comprises at least 0.2 gram of phenol per 1 gram of polypeptide copolymer. In some embodiments, the mixture comprises at least 0.4 gram of phenol per 1 gram of polypeptide copolymer. In some embodiments, the mixture comprises at least 0.6 gram of phenol per 1 gram of polypeptide copolymer.

Without being bound by any particular theory, it is believed that it is advantageous for the bromine scavenger (e.g., phenol) to be present in a concentration higher than the tyrosine residues in the polypeptide, so as to facilitate reaction of free bromine with the bromine scavenger rather than the tyrosine, once hydrogen bromide is added to the reaction mixture.

Thus, in some embodiments of any of the embodiments described herein, the mixture is such that a molar ratio of the bromine scavenger (e.g., phenol) to tyrosine residues in the polypeptide copolymer is at least 1:1 (bromine scavenger: tyrosine residue). In some embodiments, a molar ratio of bromine scavenger (e.g., phenol) to tyrosine residues is at least 1.5:1. In some embodiments, a molar ratio of bromine scavenger (e.g., phenol) to tyrosine residues is at least 2:1. In some embodiments, a molar ratio of bromine scavenger (e.g., phenol) to tyrosine residues is at least 2.5:1. In some embodiments, a molar ratio of bromine scavenger (e.g., phenol) to tyrosine residues is at least 3:1. In some embodiments, a molar ratio of bromine scavenger (e.g., phenol) to tyrosine residues is at least 4:1. In some embodiments, a molar ratio of bromine scavenger (e.g., phenol) to tyrosine residues is at least 5:1. In some embodiments, a molar ratio of bromine scavenger (e.g., phenol) to tyrosine residues is at least 6:1. In some embodiments, a molar ratio of bromine scavenger (e.g., phenol) to tyrosine residues is at least 8:1. In some embodiments, a molar ratio of bromine scavenger (e.g., phenol) to tyrosine residues is at least 10:1.

Without being bound by any particular theory, it is believed that it is desirable to avoid using more bromine scavenger (e.g., phenol) than is necessary to obtain a satisfactory result, so as to reduce costs and to obtain a relatively high concentration of the product of the reaction (i.e., a partially protected polypeptide copolymer).

Thus, in some embodiments of any of the embodiments described herein, the mixture of bromine scavenger (e.g., phenol) and protected polypeptide copolymer comprises no more than 1 gram of phenol per 1 gram of polypeptide copolymer. In some embodiments, a weight ratio of phenol to polypeptide copolymer is in a range of from 1:1 to 1:15 (phenol: polypeptide). In some embodiments, a weight ratio of phenol to polypeptide copolymer is in a range of from 1:1 to 1:10 (phenol: polypeptide). In some embodiments, a weight ratio of phenol to polypeptide copolymer is in a range of from 1:1 to 1:5 (phenol: polypeptide). In some embodiments, a weight ratio of phenol to polypeptide copolymer is in a range of from 1:1 to 0.4:1 (phenol: polypeptide). In some embodiments, a weight ratio of phenol to polypeptide copolymer is in a range of from 1:1 to 0.6:1 (phenol: polypeptide).

In some embodiments of any of the embodiments described herein, the mixture comprises no more than 0.8 gram of phenol per 1 gram of polypeptide copolymer. In some embodiments, a weight ratio of phenol to polypeptide copolymer is in a range of from 0.8:1 to 1:15 (phenol: polypeptide). In some embodiments, a weight ratio of phenol to polypeptide copolymer is in a range of from 0.8:1 to 1:10 (phenol: polypeptide). In some embodiments, a weight ratio of phenol to polypeptide copolymer is in a range of from 0.8:1 to 1:5 (phenol: polypeptide). In some embodiments, a weight ratio of phenol to polypeptide copolymer is in a range of from 0.8:1 to 0.4:1 (phenol: polypeptide). In some embodiments, a weight ratio of phenol to polypeptide copolymer is in a range of from 0.8:1 to 0.6:1 (phenol: polypeptide).

In some embodiments of any of the embodiments described herein, the mixture comprises no more than 0.6 gram of phenol per 1 gram of polypeptide copolymer. In some embodiments, a weight ratio of phenol to polypeptide copolymer is in a range of from 0.6:1 to 1:15 (phenol: polypeptide). In some embodiments, a weight ratio of phenol to polypeptide copolymer is in a range of from 0.6:1 to 1:10 (phenol: polypeptide). In some embodiments, a weight ratio of phenol to polypeptide copolymer is in a range of from 0.6:1 to 1:5 (phenol: polypeptide). In some embodiments, a weight ratio of phenol to polypeptide copolymer is in a range of from 0.6:1 to 0.4:1 (phenol: polypeptide).

In some embodiments of any of the embodiments described herein, the mixture comprises no more than 0.4 gram of phenol per 1 gram of polypeptide copolymer. In some embodiments, a weight ratio of phenol to polypeptide copolymer is in a range of from 0.4:1 to 1:15 (phenol: polypeptide). In some embodiments, a weight ratio of phenol to polypeptide copolymer is in a range of from 0.4:1 to 1:10 (phenol: polypeptide). In some embodiments, a weight ratio of phenol to polypeptide copolymer is in a range of from 0.4:1 to 1:5 (phenol: polypeptide).

In exemplary embodiments, the mixture comprises about 1.1 gram of phenol per 5 grams of protected polypeptide copolymer.

A maximal amount of bromine scavenger (e.g., phenol) may optionally be determined relative to an amount of tyrosine residues in the polypeptide in the mixture.

In some embodiments of any of the embodiments described herein, a molar ratio of the bromine scavenger (e.g., phenol) to tyrosine residues in the protected polypeptide copolymer is no more than 12:1 (bromine scavenger: tyrosine residue).

In some embodiments of any of the embodiments described herein, a molar ratio of the bromine scavenger (e.g., phenol) to tyrosine residues is no more than 10:1 (bromine scavenger: tyrosine residue). In some embodiments, the molar ratio is in a range of from 1:1 to 10:1. In some embodiments, the molar ratio is in a range of from 1.5:1 to 10:1. In some embodiments, the molar ratio is in a range of from 2:1 to 10:1. In some embodiments, the molar ratio is in a range of from 2.5:1 to 10:1. In some embodiments, the molar ratio is in a range of from 3:1 to 10:1. In some embodiments, the molar ratio is in a range of from 4:1 to 10:1. In some embodiments, the molar ratio is in a range of from 5:1 to 10:1.

In some embodiments of any of the embodiments described herein, a molar ratio of the bromine scavenger (e.g., phenol) to tyrosine residues is no more than 8:1 (bromine scavenger: tyrosine residue). In some embodiments, the molar ratio is in a range of from 1:1 to 8:1. In some embodiments, the molar ratio is in a range of from 1.5:1 to 8:1. In some embodiments, the molar ratio is in a range of from 2:1 to 8:1. In some embodiments, the molar ratio is in a range of from 2.5:1 to 8:1. In some embodiments, the molar ratio is in a range of from 3:1 to 8:1. In some embodiments, the molar ratio is in a range of from 4:1 to 8:1. In some embodiments, the molar ratio is in a range of from 5:1 to 8:1.

In some embodiments of any of the embodiments described herein, a molar ratio of the bromine scavenger (e.g., phenol) to tyrosine residues is no more than 6:1 (bromine scavenger: tyrosine residue). In some embodiments, the molar ratio is in a range of from 1:1 to 6:1. In some embodiments, the molar ratio is in a range of from 1.5:1 to 6:1. In some embodiments, the molar ratio is in a range of from 2:1 to 6:1. In some embodiments, the molar ratio is in a range of from 2.5:1 to 6:1. In some embodiments, the molar ratio is in a range of from 3:1 to 6:1. In some embodiments, the molar ratio is in a range of from 4:1 to 6:1.

In some embodiments of any of the embodiments described herein, a molar ratio of the bromine scavenger (e.g., phenol) to tyrosine residues is no more than 4:1 (bromine scavenger: tyrosine residue). In some embodiments, the molar ratio is in a range of from 1:1 to 4:1. In some embodiments, the molar ratio is in a range of from 1.5:1 to 4:1. In some embodiments, the molar ratio is in a range of from 2:1 to 4:1. In some embodiments, the molar ratio is in a range of from 2.5:1 to 4:1. In some embodiments, the molar ratio is in a range of from 3:1 to 4:1.

In some embodiments of any of the embodiments described herein, the mixture of the protected polypeptide copolymer and the bromine scavenger (e.g., phenol) is devoid of (as described herein) hydrogen bromide and/or free bromine.

The contacting of the bromine scavenger with a protected polypeptide copolymer to form the mixture (according to any of the respective embodiments described herein) may be effected by placing the bromine scavenger and protected polypeptide copolymer in a reactor (e.g., a reactor suitable for use in a large-scale preparation, according to any of the respective embodiments described herein). In some embodiments, contacting of the bromine scavenger with a protected polypeptide copolymer to form the mixture comprises stirring the bromine scavenger and protected polypeptide copolymer (e.g., in a reactor described herein). The bromine scavenger and the protected polypeptide copolymer can be placed in the reactor at any order, such that in some embodiments of any of the embodiments described herein, the mixture is formed by placing the bromine scavenger into the reactor and then placing the protected polypeptide copolymer in the reactor (e.g., a reactor according to any of the respective embodiments described herein), and/or placing the protected polypeptide copolymer into the reactor and then placing the bromine scavenger in the reactor (e.g., a reactor according to any of the respective embodiments described herein); and/or placing the bromine scavenger and protected polypeptide copolymer in the reactor (e.g., a reactor according to any of the respective embodiments described herein) simultaneously.

Deprotection and Depolymerization with Hydrogen Bromide:

The hydrogen bromide according to any one of the embodiments described in this section may be used in combination with a polypeptide copolymer according to any one of the embodiments in the section regarding the polypeptide copolymer, and with the bromine scavenger in accordance with any one of the embodiments described in the section regarding a bromine scavenger (except when incompatible).

As described herein, a solution of hydrogen bromide in acetic acid is contacted with a mixture of a protected polypeptide copolymer (according to any one of the embodiments described herein with respect to the polypeptide copolymer) and the bromine scavenger (according to any one of the embodiments described herein with respect to bromine scavenger). Hydrogen bromide is effective at deprotecting caroboxylate-protected glutamate residues (e.g., in the form of a glutamate ester), thus converting them to glutamic acid residues. In addition, acetate may optionally serve as a counter-ion in a pharmaceutically acceptable salt of the polypeptide copolymer, as described herein.

In some embodiments of any of the embodiments described herein, the solution of hydrogen bromide is not pretreated with a bromine scavenger (e.g., a bromine scavenger, optionally a phenol, according to any one of the embodiments described herein) prior to contact with the mixture.

As used herein, the term “pretreated” refers to contact between hydrogen bromide and a bromine scavenger under conditions, and for a sufficient time period, which results in reduction of free bromine levels in the hydrogen bromide by at least 50%.

In some embodiments of any of the embodiments described herein, the hydrogen bromide solution described herein is devoid of a bromine scavenger (e.g., phenol), as the terms “devoid of” and “bromine scavenger” are defined herein), prior to contacting the hydrogen bromide solution with the mixture containing the bromine scavenger as described herein.

In some embodiments, the weight percentage of bromine scavenger (e.g., phenol) in the hydrogen bromide solution is no more than 0.03%. In some embodiments, the weight percentage of bromine scavenger in the hydrogen bromide solution is no more than 0.01%. In some embodiments, the weight percentage of bromine scavenger in the hydrogen bromide solution is no more than 0.003%. In some embodiments, the weight percentage of bromine scavenger in the hydrogen bromide solution is no more than 0.001%. In some embodiments, the weight percentage of bromine scavenger in the hydrogen bromide solution is no more than 0.0003%. In some embodiments, the weight percentage of bromine scavenger in the hydrogen bromide solution is no more than 0.0001%.

In embodiments wherein the hydrogen bromide solution is not pretreated with a bromine scavenger such as phenol, the free bromine concentration in the hydrogen bromide solution may be relatively high. However, as described herein, the process described herein allows for the use of such hydrogen bromide solutions.

In some embodiments of any of the embodiments described herein, a concentration of free bromine in the hydrogen bromide solution is at least 0.1 weight percent. In some embodiments, the concentration of free bromine is at least 0.2 weight percent. In some embodiments, the concentration of free bromine is at least 0.4 weight percent. In some embodiments, the concentration of free bromine is at least 0.6 weight percent. In some embodiments, the concentration of free bromine is at least 0.8 weight percent. In some embodiments, the concentration of free bromine is at least 1 weight percent.

In some embodiments of any of the embodiments described herein, a concentration of hydrogen bromide in the (acetic acid) solution is in a range of from 10 to 40 weight percents. In some embodiments, the concentration is in a range of from 10 to 36 weight percents. In some embodiments, the concentration is in a range of from 14 to 36 weight percents. In some embodiments, the concentration is in a range of from 18 to 36 weight percents. In some embodiments, the concentration is in a range of from 21 to 36 weight percents. In some embodiments, the concentration is in a range of from 24 to 36 weight percents. In some embodiments, the concentration is in a range of from 27 to 36 weight percents. In some embodiments, the concentration is in a range of from 30 to 36 weight percents. In exemplary embodiments, the concentration is about 33 weight percents.

In some embodiments of any of the embodiments described herein, the hydrogen bromide solution and the bromine scavenger- and polypeptide-containing mixture described herein are contacted in a proportion selected such that the solution comprises at least 2 grams hydrogen bromide (that is, 2 grams of hydrogen bromide per se, not including the weight of the acetic acid) per 1 gram of the mixture. In some embodiments, the mixture and solution are contacted in a weight ratio of at least 3 grams hydrogen bromide per 1 gram of the mixture. In some embodiments, the mixture and solution are contacted in a ratio of at least 4 grams hydrogen bromide per 1 gram of the mixture. In some embodiments, the mixture and solution are contacted in a ratio of at least 5 grams hydrogen bromide per 1 gram of the mixture. In some embodiments, the mixture and solution are contacted in a ratio of at least 6 grams hydrogen bromide per 1 gram of the mixture. In some embodiments, the mixture and solution are contacted in a ratio of at least 7 grams hydrogen bromide per 1 gram of the mixture. In some embodiments, the mixture and solution are contacted in a ratio of at least 8 grams hydrogen bromide per 1 gram of the mixture.

It is to be understood that in calculating the weight of the mixture, all ingredients of the mixture are considered, including any ingredients (if present) other than the protected polypeptide copolymer and bromine scavenger described herein.

In some embodiments of any of the embodiments described herein, the hydrogen bromide solution and the bromine scavenger- and polypeptide-containing mixture described herein are contacted in a proportion selected such that a molar ratio of the bromine scavenger (e.g., phenol) to hydrogen bromide is at least 1:80 (bromine scavenger: hydrogen bromide), that is, the mixture comprises at least 1 mole of bromine scavenger (e.g., as described herein) per 80 moles hydrogen bromide. In some embodiments, a molar ratio of the bromine scavenger (e.g., phenol) to hydrogen bromide is at least 1:60. In some embodiments, a molar ratio of the bromine scavenger (e.g., phenol) to hydrogen bromide is at least 1:40. In some embodiments, a molar ratio of the bromine scavenger (e.g., phenol) to hydrogen bromide is at least 1:20.

In some embodiments of any of the embodiments described herein, the hydrogen bromide solution and a phenol- and polypeptide-containing mixture described herein are contacted in a proportion selected such that the mixture comprises at least 1 gram of phenol (e.g., as described herein) per 75 grams hydrogen bromide. In some embodiments, a weight ratio of the phenol to hydrogen bromide is at least 1 gram phenol per 60 grams hydrogen bromide. In some embodiments, the ratio is at least 1 gram phenol per 50 grams hydrogen bromide. In some embodiments, the ratio is at least 1 gram phenol per 40 grams hydrogen bromide. In some embodiments, the ratio is at least 1 gram phenol per 30 grams hydrogen bromide. In some embodiments, the ratio is at least 1 gram phenol per 25 grams hydrogen bromide. In some embodiments, the ratio is at least 1 gram phenol per 20 grams hydrogen bromide. In some embodiments, the ratio is at least 1 gram phenol per 15 grams hydrogen bromide.

Without being bound by any particular theory, it is believed that a ratio of bromine scavenger (e.g., phenol) to hydrogen bromide as described herein provides a sufficient amount of phenol to react effectively with free bromine impurities in the hydrogen bromide, and thereby inhibit bromination of tyrosine residues.

In some embodiments of any of the embodiments described herein, the hydrogen bromide solution and the phenol- and polypeptide-containing mixture described herein are contacted in a proportion selected such that a weight ratio of hydrogen bromide to the mixture is at least 2:1 (hydrogen bromide: mixture), and a weight ratio of phenol to hydrogen bromide is at least 1:75 (phenol: hydrogen bromide). In some embodiments, a weight ratio of hydrogen bromide to the mixture is at least 2:1, and a weight ratio of phenol to hydrogen bromide is at least 1:60. In some embodiments, a weight ratio of hydrogen bromide to the mixture is at least 2:1, and a weight ratio of phenol to hydrogen bromide is at least 1:50. In some embodiments, a weight ratio of hydrogen bromide to the mixture is at least 2:1, and a weight ratio of phenol to hydrogen bromide is at least 1:40. In some embodiments, a weight ratio of hydrogen bromide to the mixture is at least 2:1, and a weight ratio of phenol to hydrogen bromide is at least 1:30. In some embodiments, a weight ratio of hydrogen bromide to the mixture is at least 2:1, and a weight ratio of phenol to hydrogen bromide is at least 1:25. In some embodiments, a weight ratio of hydrogen bromide to the mixture is at least 2:1, and a weight ratio of phenol to hydrogen bromide is at least 1:20. In some embodiments, a weight ratio of hydrogen bromide to the mixture is at least 2:1, and a weight ratio of phenol to hydrogen bromide is at least 1:15.

In some embodiments of any of the embodiments described herein, the hydrogen bromide solution and the phenol- and polypeptide-containing mixture described herein are contacted in a proportion selected such that a weight ratio of hydrogen bromide to the mixture is at least 3:1 (hydrogen bromide: mixture), and a weight ratio of phenol to hydrogen bromide is at least 1:75 (phenol: hydrogen bromide). In some embodiments, a weight ratio of hydrogen bromide to the mixture is at least 3:1, and a weight ratio of phenol to hydrogen bromide is at least 1:60. In some embodiments, a weight ratio of hydrogen bromide to the mixture is at least 3:1, and a weight ratio of phenol to hydrogen bromide is at least 1:50. In some embodiments, a weight ratio of hydrogen bromide to the mixture is at least 3:1, and a weight ratio of phenol to hydrogen bromide is at least 1:40. In some embodiments, a weight ratio of hydrogen bromide to the mixture is at least 3:1, and a weight ratio of phenol to hydrogen bromide is at least 1:30. In some embodiments, a weight ratio of hydrogen bromide to the mixture is at least 3:1, and a weight ratio of phenol to hydrogen bromide is at least 1:25. In some embodiments, a weight ratio of hydrogen bromide to the mixture is at least 3:1, and a weight ratio of phenol to hydrogen bromide is at least 1:20. In some embodiments, a weight ratio of hydrogen bromide to the mixture is at least 3:1, and a weight ratio of phenol to hydrogen bromide is at least 1:15.

In some embodiments of any of the embodiments described herein, the hydrogen bromide solution and the phenol- and polypeptide-containing mixture described herein are contacted in a proportion selected such that a weight ratio of hydrogen bromide to the mixture is at least 4:1 (hydrogen bromide: mixture), and a weight ratio of phenol to hydrogen bromide is at least 1:75 (phenol: hydrogen bromide). In some embodiments, a weight ratio of hydrogen bromide to the mixture is at least 4:1, and a weight ratio of phenol to hydrogen bromide is at least 1:60. In some embodiments, a weight ratio of hydrogen bromide to the mixture is at least 4:1, and a weight ratio of phenol to hydrogen bromide is at least 1:50. In some embodiments, a weight ratio of hydrogen bromide to the mixture is at least 4:1, and a weight ratio of phenol to hydrogen bromide is at least 1:40. In some embodiments, a weight ratio of hydrogen bromide to the mixture is at least 4:1, and a weight ratio of phenol to hydrogen bromide is at least 1:30. In some embodiments, a weight ratio of hydrogen bromide to the mixture is at least 4:1, and a weight ratio of phenol to hydrogen bromide is at least 1:25. In some embodiments, a weight ratio of hydrogen bromide to the mixture is at least 4:1, and a weight ratio of phenol to hydrogen bromide is at least 1:20. In some embodiments, a weight ratio of hydrogen bromide to the mixture is at least 4:1, and a weight ratio of phenol to hydrogen bromide is at least 1:15.

In some embodiments of any of the embodiments described herein, the hydrogen bromide solution and the phenol- and polypeptide-containing mixture described herein are contacted in a proportion selected such that a weight ratio of hydrogen bromide to the mixture is at least 5:1 (hydrogen bromide: mixture), and a weight ratio of phenol to hydrogen bromide is at least 1:75 (phenol: hydrogen bromide). In some embodiments, a weight ratio of hydrogen bromide to the mixture is at least 5:1, and a weight ratio of phenol to hydrogen bromide is at least 1:60. In some embodiments, a weight ratio of hydrogen bromide to the mixture is at least 5:1, and a weight ratio of phenol to hydrogen bromide is at least 1:50. In some embodiments, a weight ratio of hydrogen bromide to the mixture is at least 5:1, and a weight ratio of phenol to hydrogen bromide is at least 1:40. In some embodiments, a weight ratio of hydrogen bromide to the mixture is at least 5:1, and a weight ratio of phenol to hydrogen bromide is at least 1:30. In some embodiments, a weight ratio of hydrogen bromide to the mixture is at least 5:1, and a weight ratio of phenol to hydrogen bromide is at least 1:25. In some embodiments, a weight ratio of hydrogen bromide to the mixture is at least 5:1, and a weight ratio of phenol to hydrogen bromide is at least 1:20. In some embodiments, a weight ratio of hydrogen bromide to the mixture is at least 5:1, and a weight ratio of phenol to hydrogen bromide is at least 1:15.

In some embodiments of any of the embodiments described herein, the hydrogen bromide solution and the phenol- and polypeptide-containing mixture described herein are contacted in a proportion selected such that a weight ratio of hydrogen bromide to the mixture is at least 6:1 (hydrogen bromide: mixture), and a weight ratio of phenol to hydrogen bromide is at least 1:75 (phenol: hydrogen bromide). In some embodiments, a weight ratio of hydrogen bromide to the mixture is at least 6:1, and a weight ratio of phenol to hydrogen bromide is at least 1:60. In some embodiments, a weight ratio of hydrogen bromide to the mixture is at least 6:1, and a weight ratio of phenol to hydrogen bromide is at least 1:50. In some embodiments, a weight ratio of hydrogen bromide to the mixture is at least 6:1, and a weight ratio of phenol to hydrogen bromide is at least 1:40. In some embodiments, a weight ratio of hydrogen bromide to the mixture is at least 6:1, and a weight ratio of phenol to hydrogen bromide is at least 1:30. In some embodiments, a weight ratio of hydrogen bromide to the mixture is at least 6:1, and a weight ratio of phenol to hydrogen bromide is at least 1:25. In some embodiments, a weight ratio of hydrogen bromide to the mixture is at least 6:1, and a weight ratio of phenol to hydrogen bromide is at least 1:20. In some embodiments, a weight ratio of hydrogen bromide to the mixture is at least 6:1, and a weight ratio of phenol to hydrogen bromide is at least 1:15.

In some embodiments of any of the embodiments described herein, the hydrogen bromide solution and the phenol- and polypeptide-containing mixture described herein are contacted in a proportion selected such that a weight ratio of hydrogen bromide to the mixture is at least 8:1 (hydrogen bromide: mixture), and a weight ratio of phenol to hydrogen bromide is at least 1:75 (phenol: hydrogen bromide). In some embodiments, a weight ratio of hydrogen bromide to the mixture is at least 8:1, and a weight ratio of phenol to hydrogen bromide is at least 1:60. In some embodiments, a weight ratio of hydrogen bromide to the mixture is at least 8:1, and a weight ratio of phenol to hydrogen bromide is at least 1:50. In some embodiments, a weight ratio of hydrogen bromide to the mixture is at least 8:1, and a weight ratio of phenol to hydrogen bromide is at least 1:40. In some embodiments, a weight ratio of hydrogen bromide to the mixture is at least 8:1, and a weight ratio of phenol to hydrogen bromide is at least 1:30. In some embodiments, a weight ratio of hydrogen bromide to the mixture is at least 8:1, and a weight ratio of phenol to hydrogen bromide is at least 1:25. In some embodiments, a weight ratio of hydrogen bromide to the mixture is at least 8:1, and a weight ratio of phenol to hydrogen bromide is at least 1:20. In some embodiments, a weight ratio of hydrogen bromide to the mixture is at least 8:1, and a weight ratio of phenol to hydrogen bromide is at least 1:15.

The hydrogen bromide and protected polypeptide copolymer are allowed to react for a time period sufficient to result in substantial removal of carboxylate-protecting groups.

In some embodiments of any of the embodiments described herein, the reaction time is at least about 10 hours, optionally at least 20 hours.

In some embodiments, the time period is about 10 hours. In some embodiments, the time period is about 12 hours. In some embodiments, the time period is about 14 hours. In some embodiments, the time period is about 16 hours. In some embodiments, the time period is about 18 hours. In some embodiments, the time period is about 20 hours. In some embodiments, the time period is about 22 hours. In some embodiments, the time period is about 24 hours. In some embodiments, the time period is about 26 hours. In some embodiments, the time period is about 28 hours. In some embodiments, the time period is about 30 hours. In some embodiments, the time period is about 33 hours. In some embodiments, the time period is about 36 hours. In some embodiments, the time period is about 40 hours. In some embodiments, the time period is about 44 hours. In some embodiments, the time period is about 48 hours.

The reaction is optionally performed at about a constant temperature. In some embodiments, the temperature is at least 18° C., optionally in a range of from 18 to 30° C. In some embodiments, such a temperature is used in combination with a reaction time of at least about 10 hours, optionally at least 20 hours (e.g., a time period described hereinabove).

In some embodiments, the temperature is at least 19° C., optionally from 19 to 30° C. In some embodiments, such a temperature is used in combination with a reaction time of at least about 10 hours, optionally at least 20 hours (e.g., a time period described hereinabove).

In some embodiments, the temperature is at least 20° C., optionally from 20 to 30° C. In some embodiments, such a temperature is used in combination with a reaction time of at least about 10 hours, optionally at least 20 hours (e.g., a time period described hereinabove).

In some embodiments, the temperature is at least 21° C., optionally from 21 to 30° C. In some embodiments, such a temperature is used in combination with a reaction time of at least about 10 hours, optionally at least 20 hours (e.g., a time period described hereinabove).

In some embodiments, the temperature is at least 22° C., optionally from 22 to 30° C. In some embodiments, such a temperature is used in combination with a reaction time of at least about 10 hours, optionally at least 20 hours (e.g., a time period described hereinabove).

In some embodiments, the temperature is at least 23° C., optionally from 23 to 30° C. In some embodiments, such a temperature is used in combination with a reaction time of at least about 10 hours, optionally at least 20 hours (e.g., a time period described hereinabove).

In some embodiments, the temperature is at least 24° C., optionally from 24 to 30° C. In some embodiments, such a temperature is used in combination with a reaction time of at least about 10 hours, optionally at least 20 hours (e.g., a time period described hereinabove).

In some embodiments, the temperature is at least 25° C., optionally from 25 to 30° C. In some embodiments, such a temperature is used in combination with a reaction time of at least about 10 hours, optionally at least 20 hours (e.g., a time period described hereinabove).

In some embodiments, the temperature is at least 26° C., optionally from 26 to 30° C. In some embodiments, such a temperature is used in combination with a reaction time of at least about 10 hours, optionally at least 20 hours (e.g., a time period described hereinabove).

In some embodiments, the temperature is at least 27° C., optionally from 27 to 30° C. In some embodiments, such a temperature is used in combination with a reaction time of at least about 10 hours, optionally at least 20 hours (e.g., a time period described hereinabove).

In some embodiments, the temperature is at least 28° C., optionally from 28 to 30° C. In some embodiments, such a temperature is used in combination with a reaction time of at least about 10 hours, optionally at least 20 hours (e.g., a time period described hereinabove).

In some embodiments, such a temperature is used in combination with a reaction time of at least about 10 hours, optionally at least 20 hours (e.g., a time period described hereinabove).

In some embodiments, the temperature is at least 29° C., optionally from 29 to 30° C. In some embodiments, such a temperature is used in combination with a reaction time of at least about 10 hours, optionally at least 20 hours (e.g., a time period described hereinabove).

In exemplary embodiments, the temperature is ambient room temperature. In some embodiments, ambient room temperature is used in combination with a reaction time of at least about 10 hours, optionally at least 20 hours (e.g., a time period described hereinabove).

At the end of the time period, the reaction may be ended by precipitation of the partially protected polypeptide copolymer. Precipitation may be effected, for example, by addition of water, optionally water cooled to a temperature of below 20° C.

The precipitated partially protected polypeptide copolymer may be separated from the mother liquor, for example, by centrifugation and/or filtration, and optionally washed.

The reaction with hydrogen bromide and/or isolation and/or storage of the partially protected polypeptide copolymer, and/or any other steps in the process described herein, are optionally performed under low-oxygen conditions, for example, in a reactor purged of oxygen using nitrogen or argon gas.

In some embodiments of any of the embodiments described herein, the contacting of the hydrogen bromide solution and the bromine scavenger- and polypeptide-containing mixture is performed in a reactor that is suitable for use in a large-scale preparation of the polypeptide copolymer. For example, in some embodiments, the reactor has a volume of at least 100 liters. In some embodiments, the reactor has a volume of at least 200 liters. In some embodiments, the reactor has a volume of at least 300 liters. In some embodiments, the reactor has a volume of at least 500 liters. In some embodiments, the reactor has a volume of at least 1000 liters.

In some embodiments of any of the embodiments described herein, the process further comprises stirring the bromine scavenger- and polypeptide-containing mixture and hydrogen bromide, for example, at a frequency of at least 10 rotations per minute, optionally at least 30 rotations per minute, and optionally at least 100 rotations per minute.

In some embodiments of any of the embodiments described herein, the reactor is configured for stirring the contents of the reactor (e.g., according to any of the respective embodiments described herein), for example, by an agitator.

In some embodiments of any of the embodiments described herein, the reactor is configured for adding the hydrogen bromide solution to the bottom of the reactor, for example, via an inlet at the bottom of the reactor or via an inlet configured to introduce the hydrogen bromide solution at the bottom of the reactor (e.g., a pipe which opens at the bottom of the reactor).

The reactor is preferably selected to be compatible with the hydrogen bromide solution, for example, having a non-metallic inner surface. The whole reactor may optionally be substantially composed of a suitable non-metallic material (e.g., glass or a polymer compatible with hydrogen bromide), or alternatively, the reactor may be lined with a suitable non-metallic material (e.g., glass or a polymer compatible with hydrogen bromide). The skilled person will be readily capable of selecting a suitable reactor. Examples of suitable reactors include, without limitation, a glass-lined reactor and a poly(tetrafluoroethylene)-lined reactor.

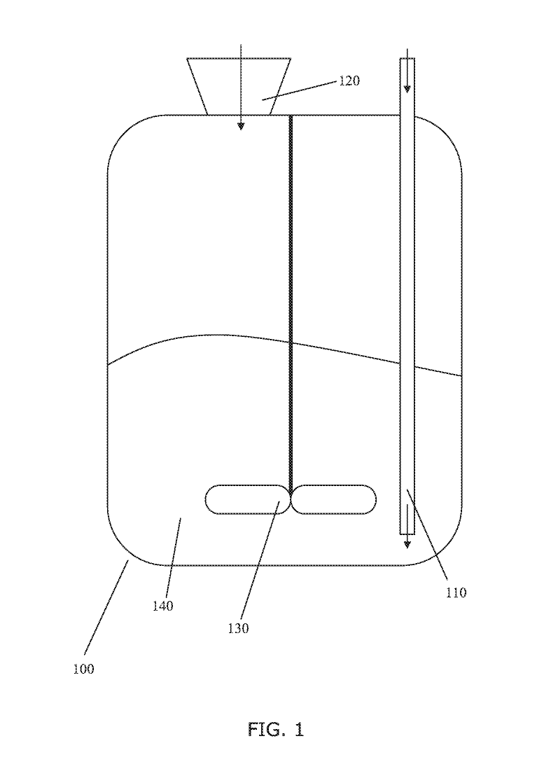

FIG. 1 depicts a reactor 100 suitable for large-scale synthesis according to some embodiments of the invention. Reactor 100 is optionally glass-lined, and has a volume of at least 100 liters, optionally at least 200 liters. Reactor 100 has an inlet 110 (optionally in a form of a deep pipe) configured to introduce a substance at the lower portion of reactor 100, through which hydrogen bromide solution may optionally be placed in the reactor, and optionally an additional inlet 120 (optionally at the upper portion thereof), through which the bromine scavenger and/or protected polypeptide copolymer may optionally be placed in the reactor. Inlet 120 may optionally be configured for inletting a solid (e.g., in a form of a funnel). Reactor 100 optionally further comprises a mechanism 130 for stirring, e.g., an agitator. Further depicted is mixture 140 of a bromine scavenger and protected polypeptide copolymer at the bottom of the volume of reactor 100.

In some embodiments of any of the embodiments described herein, a total amount of said N-carboxyanhydrides in said mixture is at least 2.5 kilograms. In some embodiments of any of the embodiments described herein, a total amount of said N-carboxyanhydrides in said mixture is at least 5 kilograms. In some embodiments of any of the embodiments described herein, a total amount of said N-carboxyanhydrides in said mixture is at least 7.5 kilograms. In some embodiments of any of the embodiments described herein, a total amount of said N-carboxyanhydrides in said mixture is at least 10 kilograms.

The contacting of the mixture comprising the bromine scavenger and polypeptide copolymer (according to any of the respective embodiments described herein) with the hydrogen bromide (according to any of the respective embodiments described herein) may be effected by adding the hydrogen bromide into a reactor containing the mixture (e.g., a reactor suitable for use in a large-scale preparation, according to any of the respective embodiments described herein). In some embodiments, contacting of the hydrogen bromide with the mixture comprises stirring the hydrogen bromide and the mixture (e.g., in a reactor described herein).

In some embodiments of any of the embodiments described herein, contacting the mixture with the hydrogen bromide comprises placing the mixture into the reactor and then placing the hydrogen bromide into the reactor (e.g., a reactor according to any of the respective embodiments described herein).

According to an aspect of some embodiments of the invention, there is provided a process of preparing a polypeptide copolymer of alanine, glutamic acid, lysine and tyrosine, or a pharmaceutically acceptable salt thereof, the process comprising:
(a) polymerizing a mixture comprising N-carboxyanhydrides of alanine, tyrosine, carboxylate-protected glutamate and an amine-protected lysine, to form a protected polypeptide copolymer (e.g., according to any of the respective embodiments described herein), a total amount of the N-carboxyanhydrides being at least 5 kilograms (e.g., according to any of the respective embodiments described herein);
(b) contacting the protected polypeptide copolymer with a bromine scavenger to form a mixture of the protected polypeptide copolymer and the bromine scavenger (e.g., according to any of the respective embodiments described herein);
(c) subsequent to (b), contacting the mixture with a solution of hydrogen bromide in acetic acid (e.g., according to any of the respective embodiments described herein), optionally in a reactor having a volume of at least 100 liters (e.g., a reactor according to any of the respective embodiments described herein), to deprotect carboxylate-protected glutamate residues in the protected polypeptide copolymer, thereby forming a partially protected polypeptide copolymer (e.g., according to any of the respective embodiments described herein); and
(d) reacting the partially protected polypeptide copolymer under conditions which effect deprotection of the amine-protected lysine (e.g., according to any of the respective embodiments described herein), to form the polypeptide copolymer or a pharmaceutically acceptable salt thereof, optionally forming at least 2 kilograms of the polypeptide copolymer, optionally at least 3 kilograms, optionally at least 4 kilograms, optionally at least 6 kilograms and optionally at least 8 kilograms of polypeptide copolymer (e.g., according to any of the respective embodiments described herein).

Such a process is also referred to herein as a “large scale process” or as a process of large scale preparation of the polypeptide copolymer.

### Deprotection of Lysine Residues:

As described hereinabove, the partially protected polypeptide copolymer obtained by deprotecting carboxylate-protected glutamic acid residues using hydrogen bromide is reacted under conditions which effect deprotection of the amine-protected lysine to form the polypeptide copolymer or a pharmaceutically acceptable salt thereof.

The skilled person will be aware of the conditions suitable for deprotection of an amine-protected lysine residues comprising any of a wide variety of amine-protecting groups known in the art.

It is expected that during the life of a patent maturing from this application many relevant amine-protecting groups and methods for deprotecting amine-protecting groups will be developed and the scope of the phrases “amine-protected” and “conditions which effect deprotection” are intended to include all such new technologies a priori.

In some embodiments (e.g., wherein the amine-protected lysine is trifluoroacetyl lysine), deprotection is effected by reaction with aqueous piperidine to form the polypeptide copolymer. This step is intended for deprotecting lysine residues by removing the N-trifluoroacetyl groups or any other amine-protecting groups from amine-protected lysine residues.

In some embodiments of any of the embodiments described herein, the aqueous piperidine comprises from 3 to 30 weight percents piperidine (relative to weight of aqueous solution). In some embodiments, the aqueous piperidine comprises from 5 to 15 weight percents piperidine. In exemplary embodiments, the aqueous piperidine comprises about 10 weight percents piperidine.

The skilled person will be capable of recognizing suitable conditions for deprotection using piperidine. Exemplary conditions are described in the Examples section herein. Suitable conditions are also described in other publications which describe preparation of glatiramer acetate, such as, for example, U.S. Pat. Nos. 7,495,072 and 7,560,100.

In some embodiments of any of the embodiments described herein, the reaction with aqueous piperidine is performed at a temperature in a range of from 15 to 30° C. In some embodiments, the reaction with aqueous piperidine is performed at a temperature in a range of from 20 to 25° C.

In some embodiments of any of the embodiments described herein, the reaction with aqueous piperidine is performed for 10 to 30 hours.

The reaction may be terminated by removing the obtained polypeptide copolymer may be separated from the reaction mixture, for example, by filtration.

### The Polypeptide Copolymer Product:

The polypeptide copolymer (or salt thereof) can optionally be purified using any suitable technique known in the art. In exemplary embodiments, the purifying comprises ultrafiltration. Suitable techniques for ultrafiltration will be known to the skilled person, and some are exemplified herein. The purified polypeptide copolymer is optionally freeze-dried.

In some embodiments of any of the embodiments described herein, the polypeptide copolymer obtained according to the process described herein is characterized by a level of brominated tyrosine residues which is less that 0.03 weight percents of the polypeptide copolymer.

According to an aspect of some embodiments of the present invention there is provides a polypeptide copolymer of alanine, glutamic acid, lysine and tyrosine, or a pharmaceutically acceptable salt thereof, according to any one of the embodiments described herein for the polypeptide copolymer, characterized in that a level of brominated tyrosine residues in the polypeptide copolymer is less than 0.03 weight percents of the polypeptide copolymer.

In some of any of these embodiments, the level of brominated tyrosine residues is less that 0.02 weight percents of the polypeptide copolymer. In some embodiments, the level of brominated tyrosine residues is less that 0.015 weight percents of the polypeptide copolymer. In some embodiments, the level of brominated tyrosine residues is less that 0.01 weight percents (100 ppm) of the polypeptide copolymer. In some embodiments, the level of brominated tyrosine residues is less that 0.0075 weight percents (75 ppm) of the polypeptide copolymer. In some embodiments, the level of brominated tyrosine residues is less that 0.005 weight percents (50 ppm) of the polypeptide copolymer. In some embodiments, the level of brominated tyrosine residues is less that 0.0025 weight percents (25 ppm) of the polypeptide copolymer. In some embodiments, the level of brominated tyrosine residues is less that 0.0015 weight percents (15 ppm) of the polypeptide copolymer. In some embodiments, the level of brominated tyrosine residues is less that 0.001 weight percents (10 ppm) of the polypeptide copolymer. In some embodiments, the level of brominated tyrosine residues is less that 0.0005 weight percents (5 ppm) of the polypeptide copolymer. In some embodiments, the level of brominated tyrosine residues is less that 0.00025 weight percents (2.5 ppm) of the polypeptide copolymer. In some embodiments, the level of brominated tyrosine residues is less that 0.0001 weight percents (1 ppm) of the polypeptide copolymer.

The level of brominated tyrosine in a polypeptide copolymer may optionally be determined by hydrolyzing the polypeptide copolymer to amino acids, and by determining the amount of brominated tyrosine relative to other amino acids by high-performance liquid chromatography (HPLC), for example, using samples of brominated tyrosine and the amino acids of the polypeptide copolymer (e.g., alanine, tyrosine, glutamic acid and/or lysine) for comparison.

In some embodiments of any of the embodiments described herein, an amount of the polypeptide copolymer prepared by the process (i.e., in a single batch, for example, a batch prepared in a reactor according to any of the respective embodiments described herein) is at least 1 kilogram. In some embodiments, the amount of polypeptide copolymer is at least 2 kilograms. In some embodiments, the amount of polypeptide copolymer is at least 3 kilograms. In some embodiments, the amount of polypeptide copolymer is at least 4 kilograms. In some embodiments, the amount of polypeptide copolymer is at least 6 kilograms. In some embodiments, the amount of polypeptide copolymer is at least 8 kilograms.

In some embodiments of any of the embodiments described herein wherein an amount of polypeptide copolymer is at least 1 kilogram, he polypeptide copolymer is prepared by a large scale process as described herein.

In some embodiments of any of the embodiments described herein wherein an amount of polypeptide copolymer is at least 1 kilogram, the level of brominated tyrosine residues is less that 0.015 weight percents of the polypeptide copolymer. In some such embodiments, the level of brominated tyrosine residues is less that 0.01 weight percents (100 ppm) of the polypeptide copolymer. In some such embodiments, the level of brominated tyrosine residues is less that 0.0075 weight percents (75 ppm) of the polypeptide copolymer. In some such embodiments, the level of brominated tyrosine residues is less that 0.005 weight percents (50 ppm) of the polypeptide copolymer. In some such embodiments, the level of brominated tyrosine residues is less that 0.0025 weight percents (25 ppm) of the polypeptide copolymer. In some such embodiments, the level of brominated tyrosine residues is less that 0.0015 weight percents (15 ppm) of the polypeptide copolymer. In some such embodiments, the level of brominated tyrosine residues is less that 0.001 weight percents (10 ppm) of the polypeptide copolymer. In some such embodiments, the level of brominated tyrosine residues is less that 0.0005 weight percents (5 ppm) of the polypeptide copolymer. In some such embodiments, the level of brominated tyrosine residues is less that 0.00025 weight percents (2.5 ppm) of the polypeptide copolymer. In some such embodiments, the level of brominated tyrosine residues is less that 0.0001 weight percents (1 ppm) of the polypeptide copolymer.

### Uses and Formulations:

The polypeptide copolymer prepared according to any one of the embodiments of the invention can be used as a pharmaceutically active agent, as previously described.

According to an aspect of some embodiments of the present invention there is provided a use of the polypeptide copolymer as described herein in the manufacture of a medicament for treating a condition treatable by the polypeptide copolymer.

According to another aspect of embodiments of the invention there is provided a polypeptide copolymer as described in any one of the embodiments herein for use in treating a condition treatable by the polypeptide copolymer.

According to another aspect of embodiments of the invention there is provided a method of treating a condition treatable by the polypeptide copolymer as described in any one of the embodiments herein, the method comprising administering the polypeptide copolymer to a subject in need thereof.

In some embodiments, the condition is a condition treatable by glatiramer acetate and optionally any polypeptide copolymer exhibiting a biological activity similar to that of glatiramer acetate (e.g., as described in the art).

Examples of conditions treatable by a polypeptide copolymer as described in any one of the embodiments herein include, without limitation, multiple sclerosis, cerebral malaria and dry age-related macular degeneration.

It is expected that during the life of a patent maturing from this application many relevant treatments and compositions utilizing a polypeptide copolymer such as described herein (e.g., glatiramer acetate and related polypeptides) will be developed and the scope of the terms “pharmaceutical composition” and “condition treatable by the polypeptide copolymer” is intended to include all such new technologies a priori.

The polypeptide copolymer prepared according to any one of the embodiments of the invention can be administered to an organism, or otherwise used, per se, or in a pharmaceutical composition where it is mixed with suitable carriers or excipients.

As used herein a “pharmaceutical composition” refers to a preparation of an active ingredient (e.g., a polypeptide copolymer described herein) with other chemical components such as physiologically suitable carriers and excipients. The purpose of a pharmaceutical composition is to facilitate administration of a compound to an organism. A pharmaceutical composition as described herein can be used as a medicament, as described herein, or in the preparation of a medicament as described herein.

Herein, the phrase “pharmaceutically acceptable carrier” refers to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

Glatiramer acetate is commonly administered by subcutaneous injection.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

In some embodiments of any of the embodiments described herein, the composition is formulated for subcutaneous injection.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions according to some embodiments of the invention include compositions wherein the polypeptide copolymer is contained in an amount effective to achieve the intended purpose. More specifically, a therapeutically effective amount means an amount of polypeptide copolymer effective to prevent, alleviate or ameliorate symptoms of a condition treatable by the polypeptide copolymer (e.g., multiple sclerosis) or prolong the survival of the subject being treated.

Determination of a therapeutically effective amount is well within the capability of those skilled in the art, especially in light of the detailed disclosure provided herein.

For any preparation according to embodiments of the invention, a therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the polypeptide copolymer described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide in vivo levels of the polypeptide copolymer sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

The skilled person will be capable of selecting a suitable dosage and treatment regimen, in view of the knowledge in the art with respect to the use of glatiramer acetate, as well as in view of the guidance provided herein.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

### Deprotection of Protected Glutamate Residues in Other Polymeric Compounds:

The sequential addition of bromine scavenger (e.g., phenol) and hydrogen bromide as described herein can be utilized in any other processes where deprotection of glutamate residue is desired or required, particularly when the glutamate-protected intermediate further comprises a phenol-containing moiety which may undesirably react with hydrogen bromide.

According to another aspect of some embodiments of the present invention, there is provided a novel process of deprotecting carboxylate-protected glutamate residues (e.g., a carboxylate-protected glutamate described herein) in a polypeptide or any other polymer comprising carboxylate-protected glutamate residues (or a pharmaceutically acceptable salt thereof). Such a process is useful, for example, for performing deprotection of carboxylate-protected glutamate residues in polypeptides or other polymeric compounds which further comprise tyrosine residues or other phenol-containing moieties, and is therefore useful in an overall synthesis of preparing such polypeptides or polymeric compounds. For example, such a process is useful for deprotecting carboxylate-protected glutamate residues in a protected polypeptide copolymer of alanine, glutamic acid, lysine and tyrosine (or a pharmaceutically acceptable salt thereof) as described herein.

γ-Benzyl glutamate residues are an exemplary form of carboxylate-protected glutamate residues.

According to some embodiments of the present invention, the process comprises:
(i) contacting the glutamate-protected polypeptide (e.g., a protected polypeptide copolymer as described herein) with a bromine scavenger (e.g., as described herein, optionally a phenol) to form a mixture of the protected polypeptide and the bromine scavenger; and
(ii) subsequent to (i), contacting the mixture with a solution of hydrogen bromide (e.g., in acetic acid), to deprotect carboxylate-protected glutamate residues in the protected polypeptide (e.g., thereby forming a partially protected polypeptide copolymer of alanine, glutamic acid, tyrosine and amine-protected lysine, as described herein).

In some embodiments, steps (i) and (ii) as described hereinabove are essentially the same as steps (b) and (c) according to other embodiments described herein.

### Miscellaneous Definitions:

As used herein throughout, the term “alkyl” refers to a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or unsubstituted. When substituted, the substituent group can be, for example, cycloalkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, phosphate, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, hydrazine, and amino, as these terms are defined herein.

A “cycloalkyl” group refers to an all-carbon monocyclic or fused ring (i.e., rings which share an adjacent pair of carbon atoms) group wherein one or more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be, for example, alkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, phosphate, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, hydrazine, and amino, as these terms are defined herein.

An “alkenyl” group refers to an unsaturated group corresponding to an alkyl group (as defined herein) which consists of at least two carbon atoms and at least one carbon-carbon double bond.

An “alkynyl” group refers to an unsaturated group corresponding to an alkyl group (as defined herein) which consists of at least two carbon atoms and at least one carbon-carbon triple bond.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, phosphate, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, hydrazine, and amino, as these terms are defined herein.

A “heteroaryl” group refers to a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or unsubstituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, phosphate, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, hydrazine, and amino, as these terms are defined herein.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. When substituted, the substituted group can be, for example, lone pair electrons, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, phosphate, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, hydrazine, and amino, as these terms are defined herein. Representative examples are piperidine, piperazine, tetrahydrofuran, tetrahydropyran, morpholine and the like.

As used herein, the terms “amine” and “amino” refer to either a —NR′R″ group, wherein R′ and R″ are selected from the group consisting of hydrogen, alkyl, cycloalkyl, heteroalicyclic (bonded through a ring carbon), aryl and heteroaryl (bonded through a ring carbon). R′ and R″ are bound via a carbon atom thereof. Optionally, R′ and R″ are selected from the group consisting of hydrogen and alkyl comprising 1 to 4 carbon atoms. Optionally, R′ and R″ are hydrogen.

An “alkoxy” group refers to both an —O-alkyl and an —O-cycloalkyl group, as defined herein.

An “aryloxy” group refers to both an —O-aryl and an —O-heteroaryl group, as defined herein.

A “thiohydroxy” or “thiol” group refers to a —SH group.

A “thioalkoxy” group refers to both an —S-alkyl group, and an —S-cycloalkyl group, as defined herein.

A “thioaryloxy” group refers to both an —S-aryl and an —S-heteroaryl group, as defined herein.

A “carbonyl” group refers to a —C(═O)—R′ group, where R′ is defined as hereinabove.

A “thiocarbonyl” group refers to a —C(═S)—R′ group, where R′ is as defined herein.

A “C-carboxy” group refers to a —C(═O)—O—R′ groups, where R′ is as defined herein.

An “O-carboxy” group refers to an R′C(═O)O—O— group, where R′ is as defined herein.

A “carboxy” or “carboxyl” encompasses both C-carboxy and O-carboxy groups, as defined herein.

An “oxo” group refers to a ═O group.

A “halo” group refers to a fluorine, chlorine, bromine or iodine atom.

A “sulfinyl” group refers to an —S(═O)—R′ group, where R′ is as defined herein.

A “sulfonyl” group refers to an —S(═O)₂—R′ group, where R′ is as defined herein.

A “sulfonate” group refers to an —S(═O)₂—O—R′ group, where R′ is as defined herein.

A “sulfate” group refers to an —O—S(═O)₂—O—R′ group, where R′ is as defined as herein.

A “sulfonamide” or “sulfonamido” group encompasses both S-sulfonamido and N-sulfonamido groups, as defined herein.

An “S-sulfonamido” group refers to a —S(═O)₂—NR′R″ group, with each of R′ and R″ as defined herein.

An “N-sulfonamido” group refers to an R′S(═O)₂—NR″ group, where each of R′ and R″ is as defined herein.

An “O-carbamyl” group refers to an —OC(═O)—NR′R″ group, where each of R′ and R″ is as defined herein.

An “N-carbamyl” group refers to an R′OC(═O)—NR″-group, where each of R′ and R″ is as defined herein.

A “carbamyl” or “carbamate” group encompasses O-carbamyl and N-carbamyl groups.

An “O-thiocarbamyl” group refers to an —OC(═S)—NR′R″ group, where each of R′ and R″ is as defined herein.

An “N-thiocarbamyl” group refers to an R′OC(═S)NR″-group, where each of R′ and R″ is as defined herein.

A “thiocarbamyl” or “thiocarbamate” group encompasses O-thiocarbamyl and N-thiocarbamyl groups.

A “C-amido” group refers to a —C(═O)—NR′R″ group, where each of R′ and R″ is as defined herein.

An “N-amido” group refers to an R′C(═O)—NR″-group, where each of R′ and R″ is as defined herein.

An “amide” group encompasses both C-amido and N-amido groups.

A “urea” group refers to an —N(R′)—C(═O)—NR″R″′ group, where each of R′ and R″ is as defined herein, and R″′ is defined as R′ and R″ are defined herein.

The term “thiourea” describes a —N(R′)—C(═S)—NR″-group, with each of R′ and R″ as defined hereinabove.

A “nitro” group refers to an —NO₂ group.

A “cyano” or “nitrile” group refers to a —C═N group.

The term “hydrazine” describes a —N(R′)—N(R″)R″′ group, with each of R′, R″ and R″′ as defined hereinabove.

The term “phosphonyl” or “phosphonate” describes a —P(═O)(OR′)(OR″) group, with R′ and R″ as defined hereinabove.

The term “phosphate” describes an —O—P(═O)(OR′)(OR″) group, with each of R′ and R″ as defined hereinabove.

As used herein, the term “about” refers to ±10% (wherein, for example, “about 50%” would mean 50 ±5% (i.e., 50 ±(10% of 50) %), and not 50 ±10%). In some embodiments of any of the embodiments described herein, the term “about” should be construed as meaning ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “a” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein, the terms “treat” and “treating” include abrogating, substantially inhibiting, slowing or reversing the progression of a condition, substantially ameliorating clinical or aesthetical symptoms of a condition or substantially preventing the appearance of clinical or aesthetical symptoms of a condition.

The term “polypeptide” as used herein throughout encompasses native polypeptides (either degradation products, synthetically synthesized polypeptides or recombinant peptides) and peptidomimetics (typically, synthetically synthesized peptides), as well as peptoids and semipeptoids which are polypeptide analogs, which may have, for example, modifications rendering the polypeptides more stable while in a body or more capable of penetrating into cells. Such modifications include, but are not limited to N terminus modification, C terminus modification, peptide bond modification, including, but not limited to, CH₂—NH, CH₂—S, CH₂—S═O, O═C—NH, CH₂—O, CH₂—CH₂, S═C—NH, CH═CH or CF═CH, backbone modifications, and residue modification.

Peptide bonds (—CO—NH—) within the peptide may be substituted, for example, by N-methylated bonds (—N(CH₃)—CO—), ester bonds (—C(R)H—C—O—O—C(R)—N—), ketomethylen bonds (—CO—CH₂—), α-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom.

These modifications can occur at any of the bonds along the polypeptide chain and even at several (2-3) at the same time.

Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

In addition to the above, the polypeptides of some embodiments of the invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmo sine, nor-valine, nor-leucine and ornithine. Furthermore, the term “amino acid” includes both D- and L-amino acids.

The polypeptides of some embodiments of the invention are preferably utilized in a linear form, although it will be appreciated that in cases where cyclization does not severely interfere with polypeptide characteristics, cyclic forms of the peptide can also be utilized.

Since the present peptides are preferably utilized in therapeutics or diagnostics which require the peptides to be in soluble form, the polypeptides of some embodiments of the invention preferably include one or more non-natural or natural polar amino acids, including but not limited to serine and threonine which are capable of increasing peptide solubility due to their hydroxy-containing side chain.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Materials and Methods

The N-carboxyanhydrides (NCA) of N-trifluoroacetyl lysine (NCA-TFA-Lys), alanine (NCA-Ala), γ-benzyl glutamate (NCA-benzyl-Glu) and tyrosine (NCA-Tyr) were obtained from Isochem.

Acetic acid was obtained from Biolab.

Diethylamine was obtained from Sigma.

Dioxane (anhydrous) was obtained from Biolab.

Hydrogen bromide (33%) in acetic acid was obtained from Chemada.

Phenol was obtained from Sigma Aldrich and Biolab.

Piperidine was obtained from Biolab.

### Example 1

### Synthesis of Glatiramer Acetate

Two liters of anhydrous dioxane were added to a 3 liter reactor under a dry atmosphere, followed by addition of NCA-TFA-Lys (40.00 grams), NCA-Ala (24.10 grams), NCA-benzyl-Glu (16.87 grams) and NCA-Tyr (8.68 grams) consecutively. The molar percentage of each N-carboxyanhydride was 45.1% NCA-Ala, 32.1% NCA-TFA-Lys, 13.8% NCA-benzyl-Glu, and 9.0% NCA-Tyr. While stifling, the reaction mixture was cooled to 20° C. When the reaction mixture was clear, 0.45 gram of DEA (diethylamine) was added at ambient temperature to initiate polymerization. The mixture became cloudy after 5-10 minutes, and bubbling of carbon dioxide was observed. The mixture was stirred at ambient temperature for about 20 hours.

The reaction mixture was thereafter transferred to 4 liters of cold water in a 10 liter container while stirring, leading to precipitation of a white solid. The product was separated from the mother liquor, and washed with water. The wet solid was dried under reduced pressure at 50° C. in a vacuum oven, yielding 63.5 grams of an intermediate (benzy- and trifluoroacetyl-protected) copolymer as a white solid.

63.5 grams of the abovementioned intermediate polymer and 14 grams of phenol were placed in a 3 liter reactor. The reactor was purged with nitrogen and then 1.0 liter of a solution of 33% hydrogen bromide in acetic acid was added at a temperature in a range of from 20° C. to 24° C. Stirring continued for 19 to 21 hours, and the reaction was then quenched by adding to water, resulting in formation of a white precipitate. The solid was thereafter separated, and washed with water, to yield a second intermediate (trifluoroacetyl-protected) polymer.

An aqueous piperidine solution (about 10 weight percents piperidine) was prepared in a 3-liter reactor by mixing 232 ml piperidine and two liters of water. Once the temperature stabilized, the second intermediate from the previous step was added at ambient temperature and the reaction mixture was stirred overnight. The reaction mixture was then filtered through a 0.45 μm PES (polyethersulfone) filter.

The crude product in solution was purified by ultrafiltration/diafiltration, first against 3-5 volumes of water, then against acetic acid solution to a pH of about 4, and then against water to a pH of 5-7. The purified solution was freeze dried, yielding 29.95 grams of glatiramer acetate as a white to off-white solid.

The obtained glatiramer acetate had brominated tyrosine residues at a level of not more than 0.02%, which represented about a 10-fold reduction as compared to glatiramer acetate obtained by the same technique without using phenol.

### Example 2

### Large-Scale Synthesis of Glatiramer Acetate

The N-carboxyanhydrides (NCAs) of L-alanine (3 kg), trifluoroacetyl-L-lysine (5 kg), γ-benzyl-L-glutamate (2.1 kg) and L-tyrosine (1.1 kg) were polymerized in a solution containing a total concentration of 0.23 M NCAs in dioxane, with diethylamine (DEA) as the polymerization initiator. The polymerization reaction was quenched with cool water, and the product was precipitated in a settling tank. The obtained polymeric intermediate was collected by gradual transfer to a centrifuge and washing with water, and spinning was continued until no liquid was observed. The precipitated polymeric intermediate was then milled using a Comil® milling apparatus.

In a second reaction step, the benzyl protecting group was removed from the glutamate side chain and the polypeptides were subjected to depolymerization (cleavage of amide bonds) to obtain polypeptides with lower mean molecular weight. 8 kg of the abovementioned polymeric intermediate, and 1.75 kg of phenol, were placed in a 250 liter glass-lined reactor, equipped with a deep pipe leading to the bottom of the reactor. A 33% solution of HBr in acetic acid (175 kg) was then added over the course of about 20 minutes through the pipe to the bottom of the reactor, and the solution was stirred by an agitator (at 140 rotations per minute) at the desired temperature (in a range of from 20° C. to 25° C.), to complete the deprotection of glutamate residues and depolymerization. The reaction was terminated by transfer to a settling tank and quenching in cool water supplemented with acetic acid. The precipitation solution was then stirred and left for phase separation. The upper liquid phase was removed and additional washing with acetic acid in water was performed. The upper phase after the additional washing was then removed, and the lower phase was transferred to a centrifuge. The obtained solid was washed with water and centrifuged.

In a third reaction step, the trifluoroacetyl protecting groups were removed from the lysine residues, using a 10% solution of piperidine in water. The solid intermediate obtained at the end of the abovementioned second reaction step was added to a 10% piperidine solution and stirred. The reaction mixture was filtered using a 0.2 μm filter.

The glatiramer acetate polypeptides were then purified by an ultrafiltration/diafiltration process, and the final molecular weight distribution was determined. The glatiramer acetate solution was filtered through a 0.22 μm PES filter, and was subjected to a series of diafiltration steps, and then the solution was further concentrated to the final target volume. The obtained glatiramer acetate was then lyophilized.

The obtained glatiramer acetate had brominated tyrosine residues at a level below 0.0014% (the quantitation limit), as determined by hydrolyzing the polypeptide copolymer to amino acids, and then determining the amount of brominated tyrosine relative to a brominated tyrosine standard by high-performance liquid chromatography (HPLC), which was compared to the amount of polypeptide copolymer in order to calculate a percentage of brominated tyrosine residues.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A process of preparing a polypeptide copolymer of alanine, glutamic acid, lysine and tyrosine, or a pharmaceutically acceptable salt thereof, the process comprising:
(a) polymerizing a mixture comprising N-carboxyanhydrides of alanine, tyrosine, carboxylate-protected glutamate and an amine-protected lysine, to form a protected polypeptide copolymer of alanine, tyrosine, carboxylate-protected glutamate, and amine-protected lysine;
(b) contacting said protected polypeptide copolymer with a bromine scavenger to form a mixture of said protected polypeptide copolymer and said bromine scavenger;
(c) subsequent to (b), contacting said mixture with a solution of hydrogen bromide in acetic acid, to deprotect carboxylate-protected glutamate residues in said protected polypeptide copolymer, thereby forming a partially protected polypeptide copolymer of alanine, glutamic acid, tyrosine and amine-protected lysine; and
(d) reacting said partially protected polypeptide copolymer under conditions which effect deprotection of said amine-protected lysine, to form the polypeptide copolymer of alanine, glutamic acid, lysine and tyrosine, or a pharmaceutically acceptable salt thereof.

2. The process of claim 1, wherein said bromine scavenger comprises a phenol.

3. The process of claim 2, wherein said phenol comprises unsubstituted phenol.

4. The process of claim 2, wherein said mixture comprises at least 1 gram of said phenol per 15 grams of said protected polypeptide copolymer.

5. The process of claim 2, wherein contacting said mixture with said solution of hydrogen bromide is performed while using a ratio of at least 1 gram of said phenol per 75 grams hydrogen bromide.

6. The process of claim 1, wherein contacting said mixture with said solution of hydrogen bromide is performed while using a ratio of at least 2 grams hydrogen bromide per 1 gram of said mixture.

7. The process of claim 1, wherein a molar ratio of said bromine scavenger to tyrosine residues in said protected polypeptide copolymer is at least 1.5:1.

8. The process of claim 1, wherein contacting said mixture with said solution of hydrogen bromide is performed while using a molar ratio of said bromine scavenger to hydrogen bromide which is at least 1:80.

9. The process of claim 1, wherein said solution of hydrogen bromide is not pretreated with a phenol prior to contact with said mixture.

10. The process of claim 1, wherein said carboxylate-protected glutamate is γ—benzyl glutamate.

11. The process of claim 1, wherein said amine-protected lysine is trifluoroacetyl lysine.

12. The process of claim 11, wherein deprotection of said trifluoroacetyl lysine is effected by reaction with aqueous piperidine.

13. The process of claim 11, wherein said mixture of said N-carboxyanhydrides comprises from 40 to 50 weight percents trifluoroacetyl lysine N-carboxyanhydride, from 22.5 to 30 weight percents alanine N-carboxyanhydride, from 15 to 22.5 weight percents γ-benzyl glutamate N-carboxyanhydride, and from 7.5 to 12.5 weight percents tyrosine N-carboxyanhydride.

14. The process of claim 13, wherein said mixture of said N-carboxyanhydrides comprises about 44.6 weight percents trifluoroacetyl lysine N-carboxyanhydride, about 26.9 weight percents alanine N-carboxyanhydride, about 18.8 weight percents γ-benzyl glutamate N-carboxyanhydride, and about 9.7 weight percents tyrosine N-carboxyanhydride.

15. The process of claim 1, wherein said polypeptide copolymer comprises alanine, glutamic acid, lysine and tyrosine residues in molar percentages of from 40 to 50% alanine, from 10 to 18% glutamic acid, from 28 to 36% lysine, and from 7 to 11% tyrosine.

16. The process of claim 15, wherein said polypeptide copolymer comprises alanine, glutamic acid, lysine and tyrosine residues in molar percentages of about 45.1% alanine, about 13.8% glutamic acid, about 32.1% lysine, and about 9.0% tyrosine.

17. The process of claim 1, wherein said polypeptide copolymer or a pharmaceutically acceptable salt thereof is glatiramer acetate.

18. The process of claim 1, further comprising purifying the polypeptide copolymer of alanine, glutamic acid, lysine and tyrosine, or a pharmaceutically acceptable salt thereof.

19. The process of claim 18, wherein said purifying comprises ultrafiltration.

20. The process of claim 1, wherein a level of brominated tyrosine residues in the polypeptide copolymer is less than 0.03 weight percents of the polypeptide copolymer.

21. The process of claim 20, wherein a level of brominated tyrosine residues in the polypeptide copolymer is less than 0.0025 weight percents of the polypeptide copolymer.

22. The process of claim 1, wherein contacting said mixture with said solution of hydrogen bromide in acetic acid is effected in a reactor having a volume of at least 100 liters.

23. The process of claim 1, wherein an amount of said polypeptide copolymer of alanine, glutamic acid, lysine and tyrosine is at least 2 kilograms.

24. The process of claim 23, wherein a total amount of said N-carboxyanhydrides in said mixture is at least 5 kilograms.

25. A process of preparing at least 2 kilograms of a polypeptide copolymer of alanine, glutamic acid, lysine and tyrosine, or a pharmaceutically acceptable salt thereof, the process comprising:
(a) polymerizing a mixture comprising N-carboxyanhydrides of alanine, tyrosine, carboxylate-protected glutamate and an amine-protected lysine, a total amount of said N-carboxyanhydrides being at least 5 kilograms, to form a protected polypeptide copolymer of alanine, tyrosine, carboxylate-protected glutamate, and amine-protected lysine;
(b) contacting said protected polypeptide copolymer with a bromine scavenger to form a mixture of said protected polypeptide copolymer and said bromine scavenger;
(c) subsequent to (b), contacting said mixture with a solution of hydrogen bromide in acetic acid in a reactor having a volume of at least 100 liters, to deprotect carboxylate-protected glutamate residues in said protected polypeptide copolymer, thereby forming a partially protected polypeptide copolymer of alanine, glutamic acid, tyrosine and amine-protected lysine; and
(d) reacting said partially protected polypeptide copolymer under conditions which effect deprotection of said amine-protected lysine, to form the polypeptide copolymer of alanine, glutamic acid, lysine and tyrosine, or a pharmaceutically acceptable salt thereof.

26. The process of claim 25, wherein a level of brominated tyrosine residues in the polypeptide copolymer is less than 0.0025 weight percents of the polypeptide copolymer.

27. A polypeptide copolymer of alanine, glutamic acid, lysine and tyrosine, or a pharmaceutically acceptable salt thereof, prepared by the process of claim 1.

28. The polypeptide copolymer of claim 27, wherein a level of brominated tyrosine residues in the polypeptide copolymer is less than 0.03 weight percents of the polypeptide copolymer.

29. A polypeptide copolymer of alanine, glutamic acid, lysine and tyrosine, or a pharmaceutically acceptable salt thereof, characterized in that a level of brominated tyrosine residues in the polypeptide copolymer is less than 0.03 weight percents of the polypeptide copolymer.

30. The polypeptide copolymer of claim 29, characterized in that a level of brominated tyrosine residues in the polypeptide copolymer is less than 0.0025 weight percents of the polypeptide copolymer.

31. A process of deprotecting carboxylate-protected glutamate residues in a protected polypeptide copolymer of alanine, tyrosine, carboxylate-protected glutamate, and amine-protected lysine, the process comprising:
(i) contacting said protected polypeptide copolymer with a bromine scavenger to form a mixture of said protected polypeptide copolymer and said bromine scavenger; and
(ii) subsequent to (i), contacting said mixture with a solution of hydrogen bromide in acetic acid, thereby deprotecting carboxylate-protected glutamate residues in said protected polypeptide copolymer, thereby forming a partially protected polypeptide copolymer of alanine, glutamic acid, tyrosine and amine-protected lysine.

32. A pharmaceutical composition comprising the polypeptide copolymer of claim 27.

33. The composition of claim 32, further comprising a pharmaceutically acceptable carrier.

34. A pharmaceutical composition comprising the polypeptide copolymer of claim 29.

35. The composition of claim 34, further comprising a pharmaceutically acceptable carrier.

36. A method of treating a medical condition treatable by polypeptide copolymer of alanine, glutamic acid, lysine and tyrosine, or a pharmaceutically acceptable salt thereof, the method comprising administering to a subject in need thereof a therapeutically effective amount of the polypeptide copolymer of claim 27.

37. A method of treating a medical condition treatable by polypeptide copolymer of alanine, glutamic acid, lysine and tyrosine, or a pharmaceutically acceptable salt thereof, the method comprising administering to a subject in need thereof a therapeutically effective amount of the polypeptide copolymer of claim 29.
